(19) United States
(12) Patent Application Publication
(10) Pub. No.: US 2022/0412950 A1
(43) Pub. Date: Dec. 29, 2022

(54) METHOD AND MEASURING DEVICE FOR MEASURING A SAMPLE, AND METHOD FOR CALIBRATING A TEST-DEVICE BATCH

(71) Applicant: Magnasense Technologies Oy, VANTAA (FI)

(72) Inventors: Mika Laitinen, VANTAA (FI); Markus Sovonen, RAJAMAKI (FI)

(21) Appl. No.: 17/778,474

(22) PCT Filed: Nov. 30, 2020
(86) PCT No.: PCT/FI2020/050807

§ 371 Date: May 20, 2022

(30) Foreign Application Priority Data
Nov. 28, 2019 (FI) 20196031

(51) Int. Cl. G01N 33/487; G01N 33/543

(52) U.S. Cl. CPC G01N 33/48771; G01N 33/54388; G01N 2470/04

(57) ABSTRACT

A method is disclosed for measuring a sample, in which the sample to be analysed is measured using a measuring device in connection with a development of a measurement of the sample. On the basis of the measurement, information concerning an usability of the measurement and/or an analysis result is formed in connection with a development of a measurement of the sample using a calibration comprising a set of criteria and the corresponding information is reported.

The invention relates to a method for measuring a sample, in which
the sample to be analysed is measured, using a measuring device, from one or more detection areas arranged on the test device's indicator part, and equipped with test chemistry for analysing the sample,
on the basis of the measurement, information is formed using calibration relating to the one or more detection areas arranged for the sample being analysed,
the information concerning the measured sample is reported.

In addition, the invention also relates to a measuring device, a computer program, computer program-product, as well as a test-device series, a data structure and data structure record, and a method for calibrating a test-device batch.

In sandwich assays, a sample containing the molecule being examined, i.e. the analyte, forms a bridge with a binder molecule (generally an antibody) in place on/fixed to the test base and with a stamped binder molecule (which is generally also an antibody) mixed with the sample. Any known stamp whatever, which can be measured (radioactive, coloured, fluorescent, magnetic, etc. stamps) can be used as the stamp.

Lateral-flow assays are one example of a sandwich assay. In lateral-flow assays the area, on which the stationary binder molecule is, is called the test line or corresponding area.

In addition to the test line, in lateral flow assays there is typically also a so-called control line or corresponding area. It is intended to produce a signal also when there is no, or insufficient analyte in the sample being analysed. The control line tells that the test is “in order” and the result is reliable. One can also refer generally to the test's usability.

The control line can be dependent on, or independent of the test line. A dependent control line is implemented in such a way that stamped binder molecules that have passed over the test line bind to it. The control line's signal/intensity, more generally the measurement result, is inversely dependent on the test line's signal. In other words, the more stamp that has bound to the test line, the less is left to bind to the control line.

An independent control line's signal does not, in turn, depend (at least specifically) on the test line's intensity. It can be implemented, for example, by using a second stamped binder molecule, which does not bind to either the test line or the analyte. A dependent control line is always located after the test line. An independent control line can, in principle, be located anywhere, even though, for example to be sure, it is generally located after the test line.

The Hook effect, i.e. the prozone phenomenon, is a phenomenon that influences in all so-called sandwich-type assays. One problem relating to known assays is, for example, that the Hook effect limits the test's dynamic operational concentration zone. Another problem is that, in addition, due to the Hook effect, very high concentrations of the analyte give a wrong result. Thus the Hook effect too can said to affect a test's usability. In addition, also the control and test line and their measurement leads to demands on the test device and also the measuring device.

Tests demanding a control line are additionally limited by the fact that they are unsuitable for use with so-called multiplexing. A multiplexing test means a test in which, for example, instead of one test line there are several, for example 5 test lines. All methods that are in any way based on a control line are unsuitable for use with such tests, as in practice each test line cannot have its own control line.

The present invention is intended to create a method, a measuring device, a computer-program product, a computer program, and a test-device series for measuring a sample. In addition, the invention is intended to create a data structure and data structure record, and further also a method for calibrating a test-device batch. Thus, the invention does not relate solely to the measurement of an unknown sample and the related equipment, but it also relates to the calibration of a measurement and test-device batch and the related equipment. The characteristic features of the various objects of the invention are stated in the accompanying Claims.

In the case of an unknown sample, i.e. one that is to be analysed, i.e. measured, the invention is based on calibration data being arranged for the measuring device and a set of criteria relating to one or more calibration functions being formed from it, which can be used to measure one or more detection areas of the test device's indicator part in connection with the development of the sample's measurement, i.e. before, for example, an end point set for the test device's measurement procedure, which can be set for the measuring device. In other words, the sample's measurement can be said to be being performed, one or more times, already when the development of the sample is in progress in the test device. On the basis of the measurement performed in this way, the usability of the sample's measurement can be determined. Based on the determination of the measurement's usability, it is also possible to control the progression of the sample's measurement on the measuring device and/or form information concerning the sample's measurement. Both of these can also take place in connection with the development of the sample's measurement, i.e. once again before the end point of the measurement procedure of the test device possibly set for the measuring device.

In the calibration method according to the invention, the signal given by the test device, i.e. the measurement variable using several different concentrations as a function of time, is in turn measured. Thus, the development functions of the concentrations are obtained. As the test develops on the test device, the measured measurement variable increases at a certain speed as time progresses. On the basis of the development functions obtained, the so-called calibration functions (standard curves) can be defined at different moments in time. The formed calibration functions with the sets of criteria related to them and defined in a set manner can be utilized in the measurement of an unknown sample by the measuring device.

The set of criteria can include at least one criterion value formed on the basis of the calibration function and related to it, together with conditions, on the basis of which the usability of the sample's measurement is defined in connection with the progression of the sample's measurement by the measuring device. In addition, if the sample's measurement is shown to be usable at the moment in time defined by the relevant calibration, on its basis the progression of the sample's measurement can be controlled and/or information can be formed concerning the sample's measurement.

If there is one criterion value with conditions, then, for example, two domains can be defined from the calibration function. If there are two (or more) criterion values with conditions, then, for example, three or more domains can be defined from the calibration function. In addition to the usability definition to be performed, the progression of the measurement can, owing to the invention, be controlled in different ways when the measurement variable is in different domains of the calibration function.

In some of the calibration function's domains, such as, for example, in the domain defined by the criterion values between them, the measurement of the sample is performed, forming and reporting, for example, its numerical analysis result. In some of the calibration function's domains, such as, for example, the area of the calibration function below the domain between the criterion values, the sample's measurement can be continued from the moment in time defined by the relevant calibration to the next moment in time defined by the calibration, which in turn corresponds again to its own calibration function. There, at the next moment in time, an examination like that above can be performed, or else the measurement can be directly performed, and a numerical analysis result of the sample formed and reported. Thus, the invention offers many different possibilities for measuring a sample, i.e. for determining its usability, and also for controlling the measurement itself.

In other words, when the calibration data, such as, for example, parameters of the calibration functions are included to a test-device batch's factory calibration, the quantitative result of a test, for example, at high sample concentrations can be reported already earlier and/or at low sample concentrations later than would otherwise be the case, on the basis of the formed sets of criteria. Due to this, compared for example to the test being measured only once, such as, for example, at the end point of the test device's measurement procedure defined and set by the measuring device, at which the reading of a test device is typically performed according to the prior art, one or more of the following exemplary advantages are gained using the invention:

1) The Hook effect can be recognized and erroneous results prevented,

2) The test's dynamic area expands upwards without the resolution suffering, and/or,

3) The test's dynamic area expands downwards without the resolution suffering.

According to some examples of embodiments, the invention can also be characterized in that
the standard curves, i.e. the calibration functions and their parameters (the calibration parameters and the criterion values and conditions belonging to the set of criteria) are defined in the test batch's factory calibration,
the measuring device is arranged to use the calibration parameters for measuring the sample and to make a measurement of the sample to be analysed, and to give a result concerning it, without requiring additional operations by the operator,
the quantitative result can be already reported to the operator very early (relative to the end point of the test device's measuring procedure, set for the measuring device), such as, for example, already at 1 min,
owing to the invention, the test device's control line need not be measured (examine the dependent control line either independently, or together with the test line), nor is the whole control line necessarily required at all (at least for the functioning of the invention).

The method according to the invention functions with both qualitative tests (on a scale of +/− or negative, positive, highly positive) and also quantitative, (the result of which is expressed, for example, in concentration units (for example 250 mg/l)). As a test permitting a quantitative implementation, the resolution of the test is very good. In it, the result is reported as a reading value. In calibration, the result can also be reported as “result”±deviation, in which the deviation depicts the resolution.

Owing to the invention, a qualitative or quantitative result can be given already at an early stage, long before the end point set for the test's measurement, but however, before the Hook effect can influence the result. Thus, owing to the invention, the sample's speed of development in one or more detection areas of the test device's indicator part no longer affects the measurement result. In addition, the operator can be notified already at an early stage if the Hook effect influences the result.

In addition, the invention's advantages are also that the invention functions in all cases (all sandwich assays—not only lateral flow, all forms of the control line—not only dependent, but also independent, and entirely without a control line). Thus, the test can be implemented by simply reading the test line, i.e. more generally, the detection area, in the case of its method, the test device and also its measuring device. For its part, this simplifies the actual measuring device. Owing to the invention it does not require a measuring part to measure the control area. A corresponding advantage is also realized in test devices. They can, if desired, be implemented without a control part. In addition, the invention does not restrict its stamp to any particular test form.

By defining functionality according to the invention beforehand already in factory calibration, the method is, compared to most other ways, considerably faster than making the definition only once a test is being run using the measuring device. Thus, the invention also simplifies and thus facilitates implementing a test in an actual test situation. It does not include several stages complicating the test, for example, in the form of calibration. These make the test laborious and thus also time-consuming. Owing to the invention there is no need to run several samples on the measuring device before measuring an actual unknown sample. It is particularly challenging to perform calibration runs in field measurements, i.e. outside the laboratory. The same is also true, for example, of testing performed in small clinics.

In the following, the invention, which is not restricted to the embodiments described in the following, is described in greater detail with reference to the accompanying figures, in which

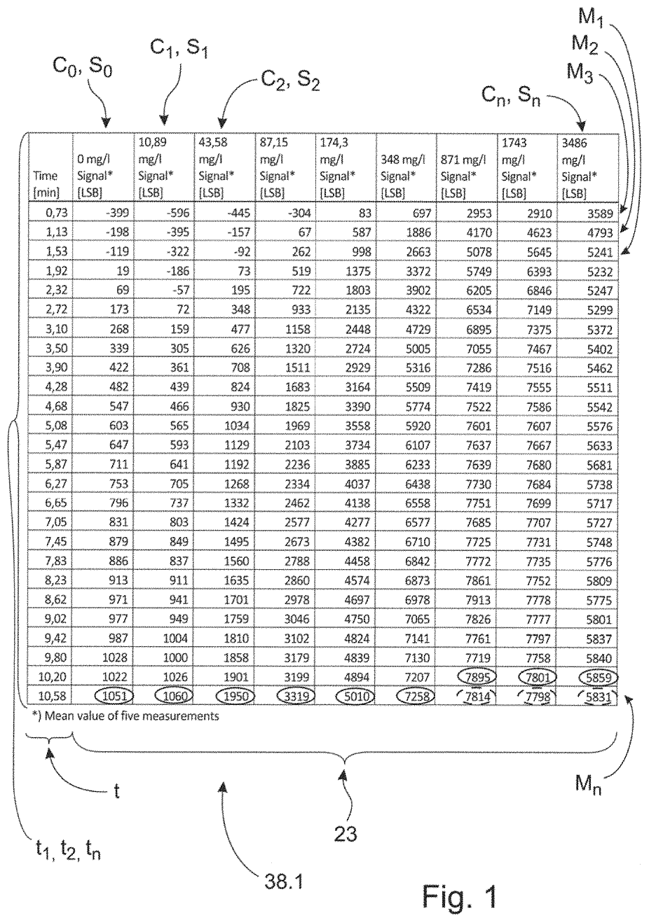

FIG. 1 shows an example of data of development curves at different concentrations, when calibrating a test,

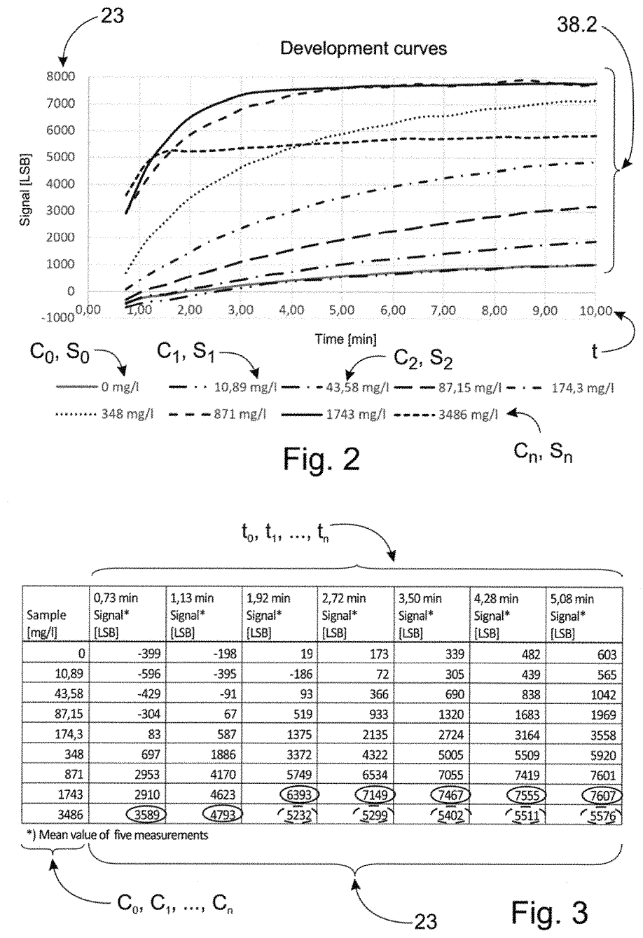

FIG. 2 shows the development curves formed from the data of FIG. 1, at different concentrations,

FIG. 3 shows the responses of different concentrations at different points in time,

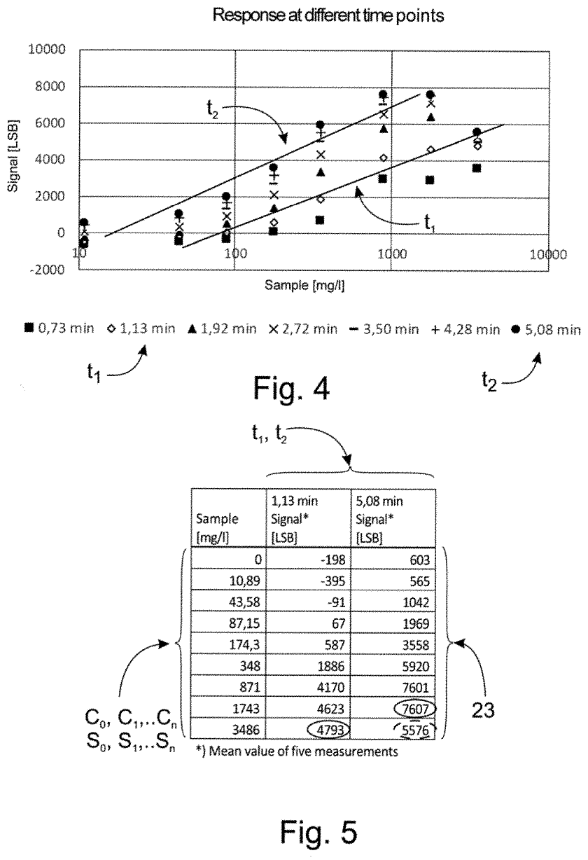

FIG. 4 shows a curve formed from FIG. 3 from responses formed at different moments in time at different concentrations,

FIG. 5 shows the response of different concentrations at two different points in time,

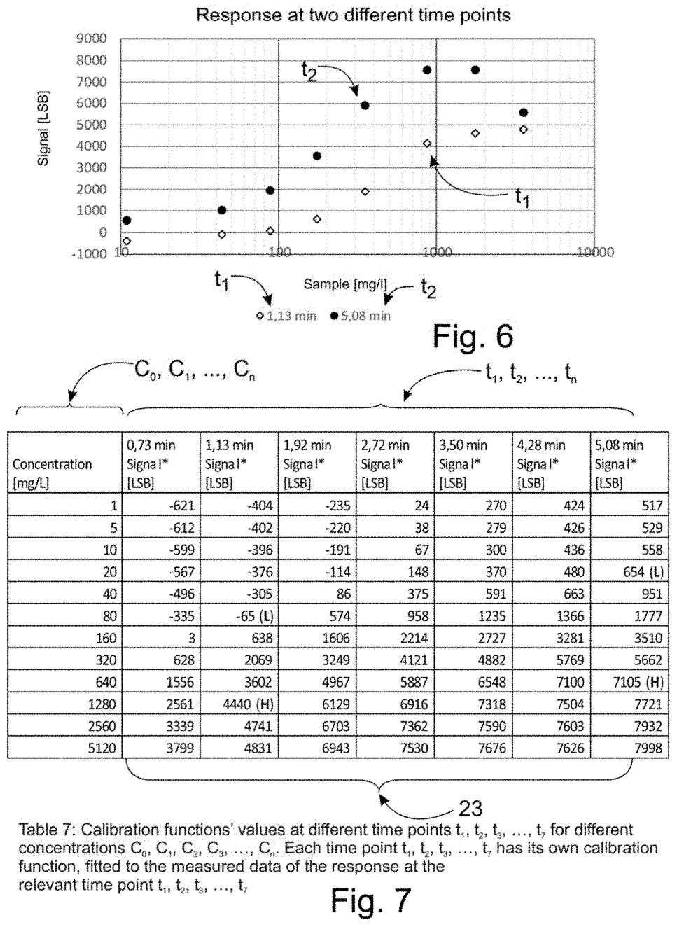

FIG. 6 shows a curve formed from FIG. 5 of the responses of different concentrations at two different points in time,

FIG. 7 shows the calibration functions' values at different points in time for different concentrations,

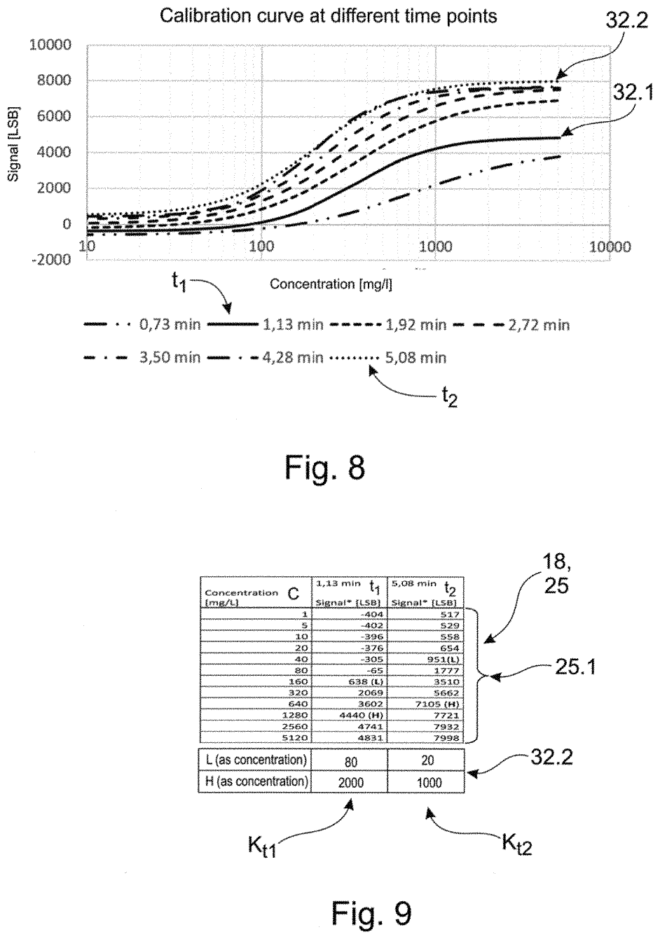

FIG. 8 shows the calibration functions of different points in time formed from the data of FIG. 7,

FIG. 9 shows the calibration functions' values at two different points in time for different concentrations and also a schematic example of a data structure for the calibration of the measuring device,

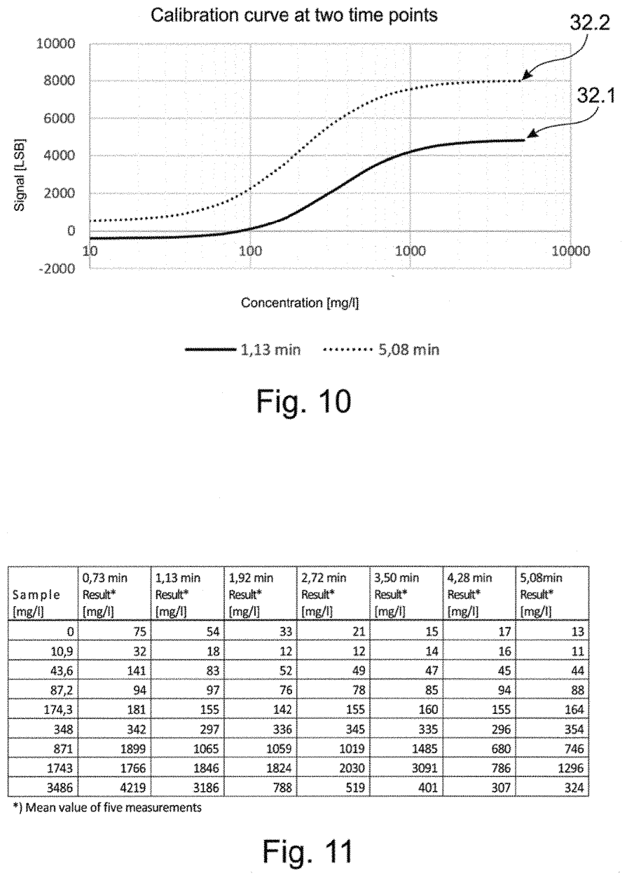

FIG. 10 shows the calibration functions of two different points in time formed from the data of FIG. 9,

FIG. 11 shows the response measurement values of each point in time, converted with the aid of a calibration function specific to the point in time to form the sample's concentration,

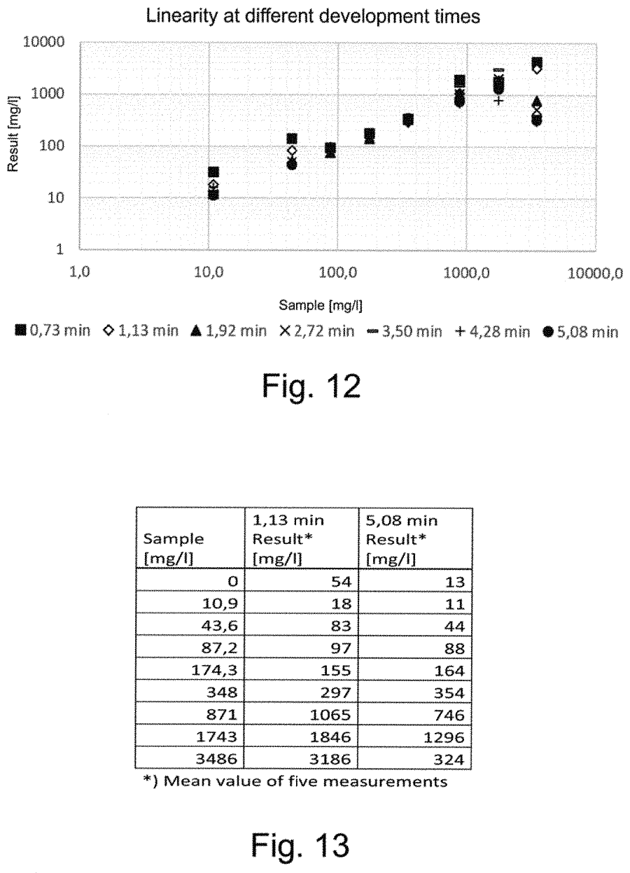

FIG. 12 shows linear curves at different points in time, formed from the data of FIG. 11,

FIG. 13 shows the response measurement values of two different points in time, converted with the aid of a calibration function specific to a point in time to form a concentration,

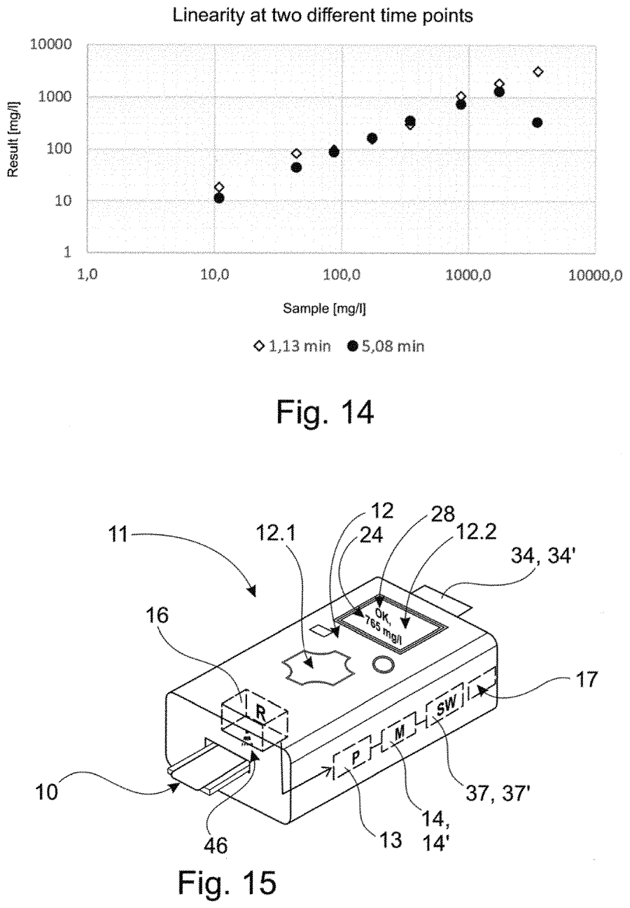

FIG. 14 shows linearity curves formed from the data of FIG. 13, at two different points in time,

FIG. 15 shows schematically an example of the measuring device,

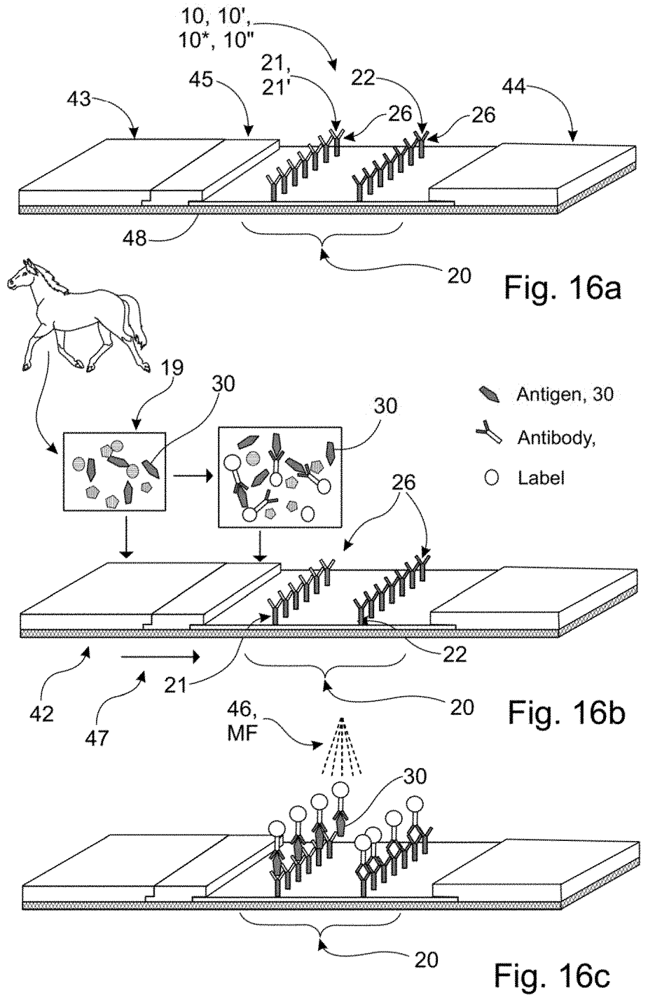

FIGS. 16a-c show an example of the test device, being now a test strip in different stages of the test,

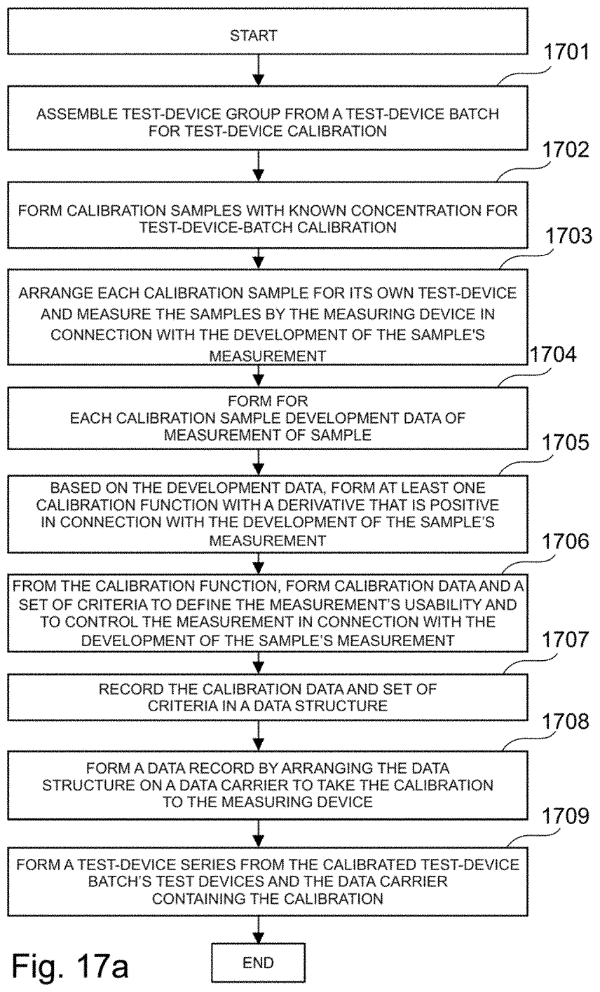

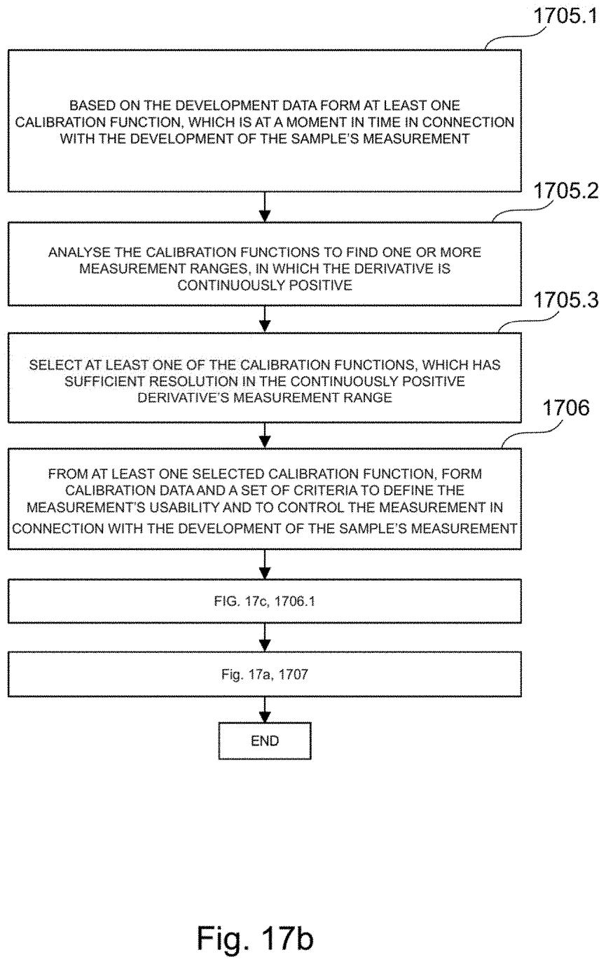

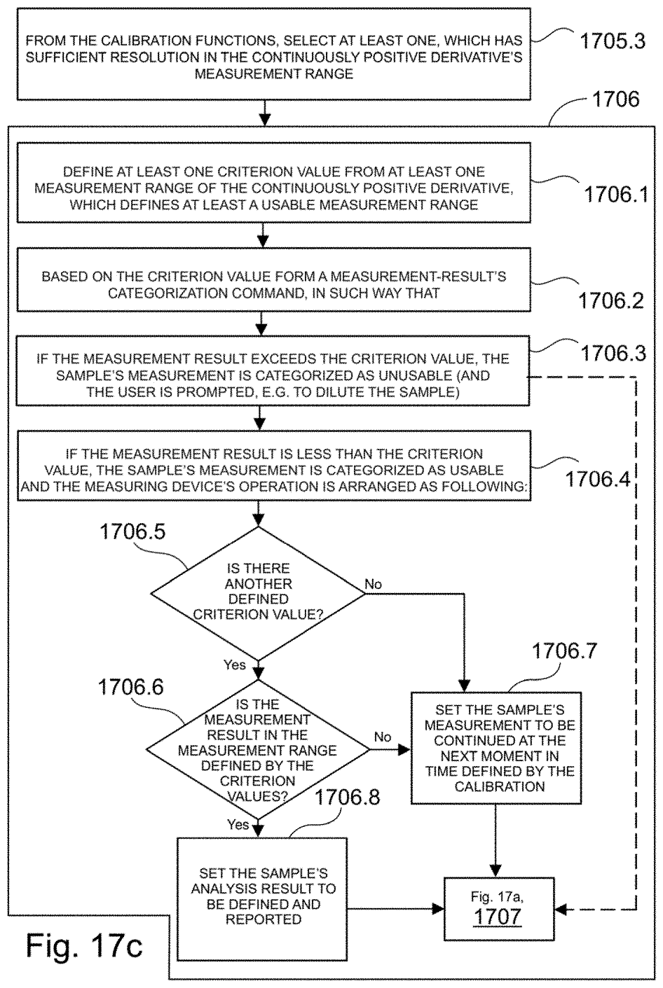

FIG. 17a shows a flow diagram of an example of the method according to the invention for performing calibration on a test-device batch,

FIG. 17b shows a flow diagram of part of the calibration in greater detail, concerning the selection of the calibration function,

FIG. 17c shows a flow diagram of part of the calibration in greater detail, concerning the criterion values belonging to the set of criteria, the categorization performed on their basis, and the forming of operating instructions corresponding to them for the calibration,

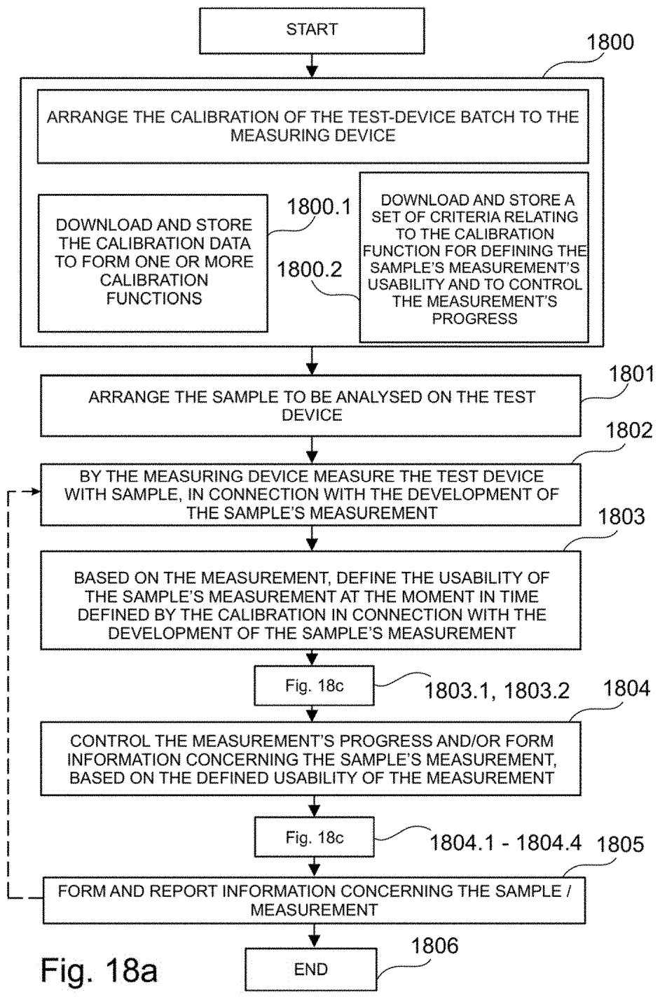

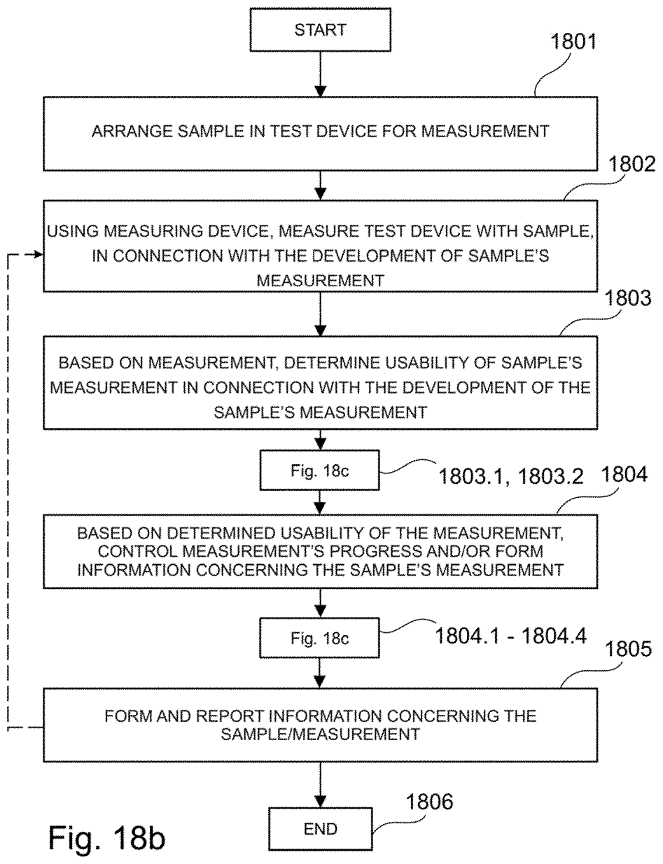

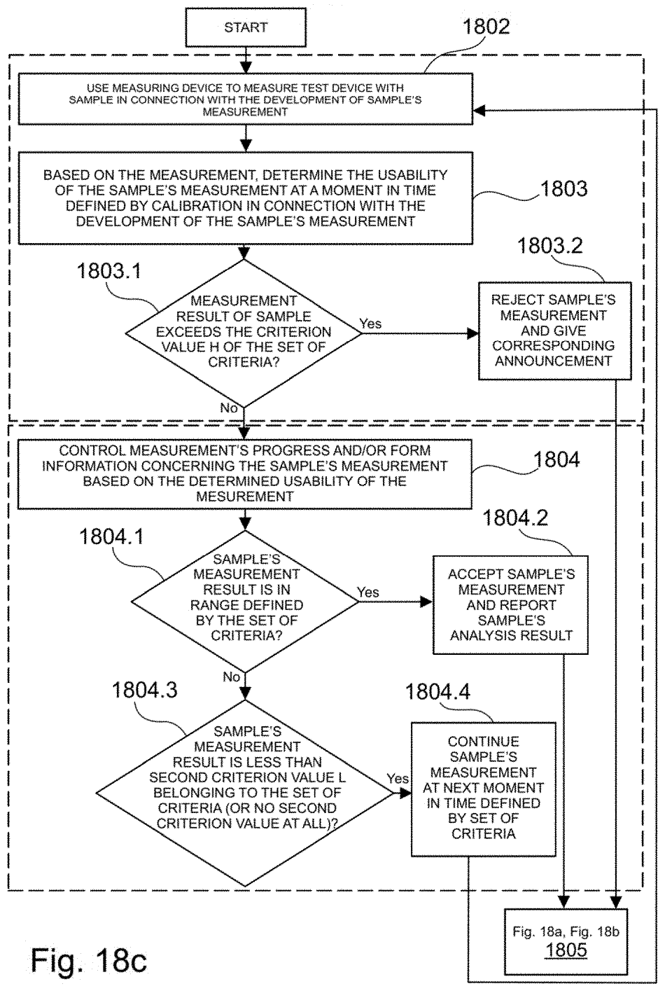

FIG. 18a shows a flow diagram of a measuring procedure using a measuring device for a sample with an unknown concentration in the case of the measurement's sub-stages, as one exemplary embodiment,

FIG. 18b shows a more generalized flow diagram of FIG. 18a, in a situation in which the calibration of the test-device series has already been taken to the measuring device,

FIG. 18c shows a flow diagram of the part of the measurement procedure of FIGS. 18a and 18b for controlling the operation of the measuring device in the case of the sub-stages, as one exemplary embodiment,

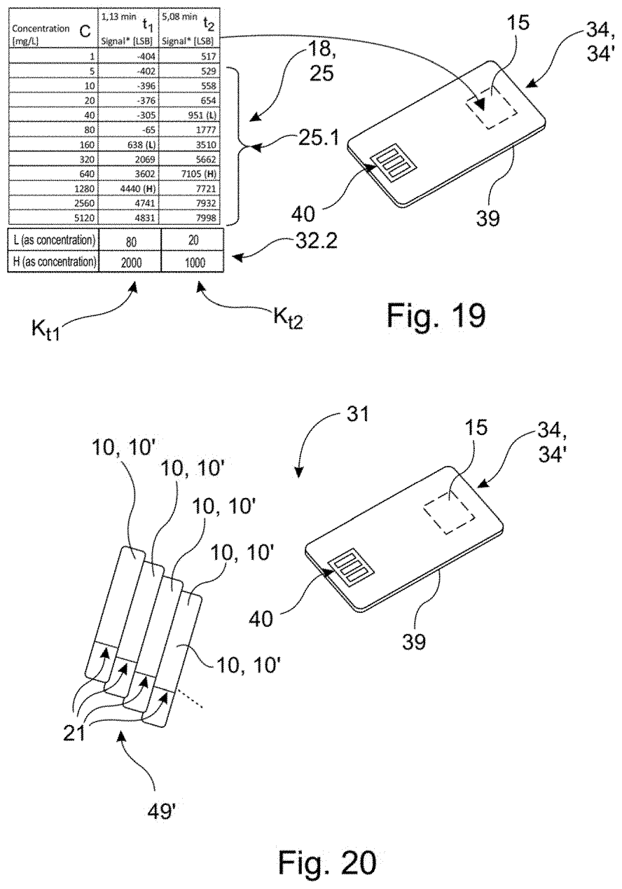

FIG. 19 shows an example of the data structure record as a rough schematic diagram,

FIG. 20 shows an example of a test-device series as a flow diagram,

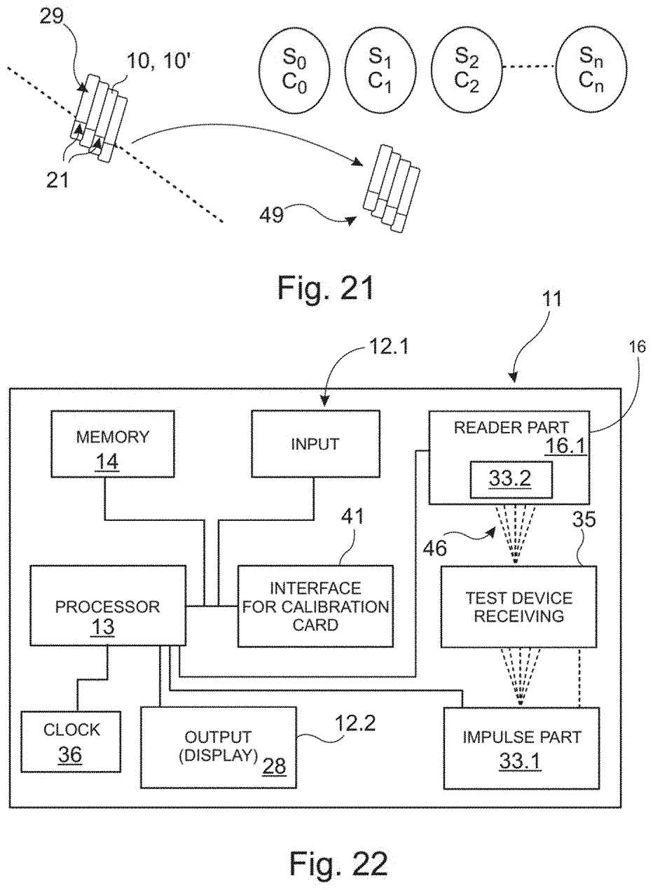

FIG. 21 shows an example of the sub-components of the calibration of a test-device batch,

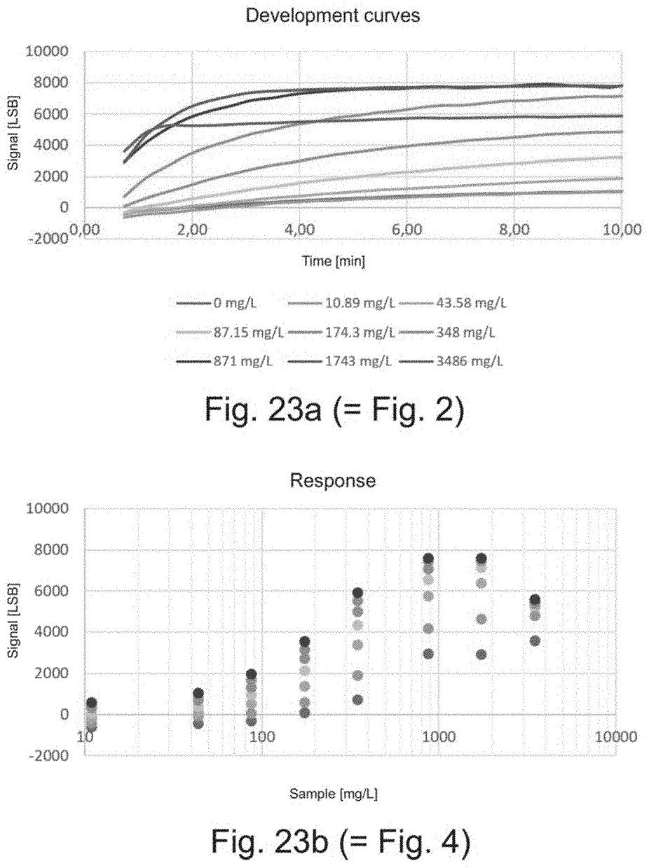

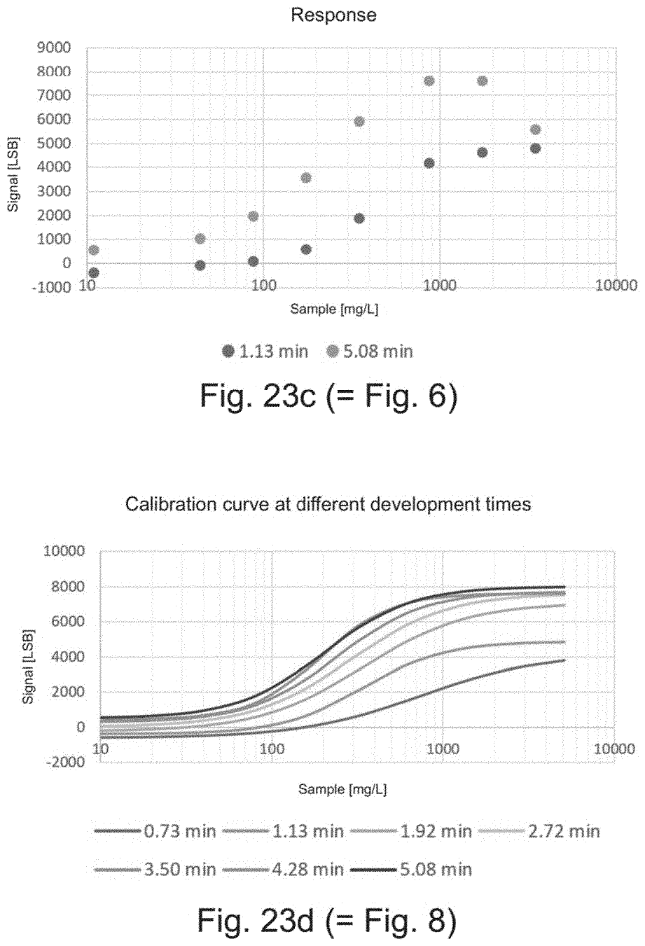

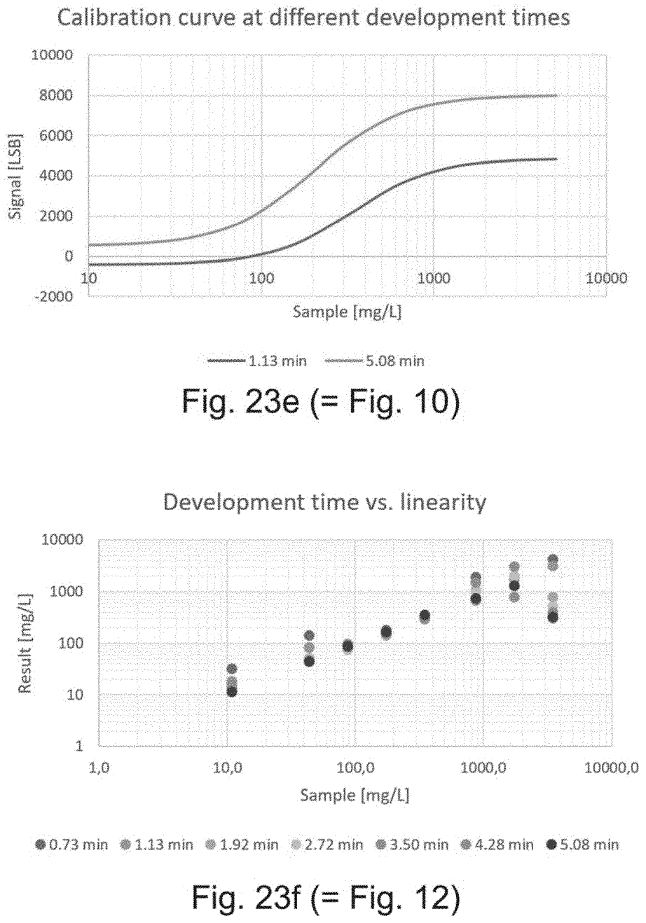

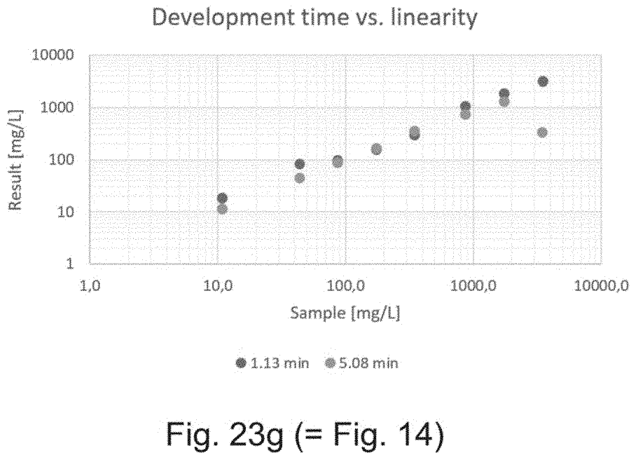

FIG. 22 shows an example of the measuring device as a schematic flow diagram, and

FIGS. 23a-g show the curves of FIGS. 2, 4, 6, 8, 10, 12, and 14 as colour images.

The invention is described first in the case of the calibration of the test and then in the performance of the actual test itself, i.e. for example, in the case of the measurement of a sample 19 with an unknown concentration, using the test device 10′ (FIGS. 16a-16c) on the measuring device 11 (FIG. 15). Thus, in the case of the description of its method, the invention can be divided, for example, into calibration and measuring stages, both methods being equally objects of the invention in the present application.

FIG. 17a shows a flow diagram of an example of the method, according to the invention, for performing calibration, on a test-device batch 29 shown schematically in FIG. 21. Here, the name test strip 10 is also used for the test device 10′. As stage 1701, a group 49 of test devices 10′, equipped with an indicator part 20 for measuring one or more analytes 30 from a sample 19, is selected from the test-device batch 29. The test-strip group 49 can be formed, for example, in an as such known manner, from a large manufacturing batch (e.g. 10000 items) of test strips 10, of which an example is shown in FIGS. 16a-16c. A sufficient sampling (e.g. 10-100 items) of test strips 10 is taken from the manufacturing batch for calibration according to the method.

The changing parameters relating to the test strips 10/reagents (test chemistry 26) arranged in the test strips 10, which require calibration of the relevant test-device batch 29 for their different manufacturing batches, are, for example, that the speed of progression of the sample and/or the reaction speed of the analyte 30 being examined in the sample 19 with the reagents 26, or in the speed of development of the sample 19, can vary between different test-strip batches.

FIG. 21 shows an example of the sub-parts relating to the calibration of a test-device batch 29. For the calibration of the test-device batch 29 also a calibration sample series is formed as stage 1702, for example, in an as such known manner. It includes a group of calibration samples S₀, S₁, S₂, . . . for performing calibration. The analyte 30 concentrations C₀, C₁, C₂, . . . in the calibration samples S₀, S₁, S₂, . . . differ from each other. The concentration C₀, C₁, C₂, . . . of each calibration sample S₀, S₁, S₂, . . . is known. The samples' S₀, S₁, S₂, . . . concentrations C₀, C₁, C₂, . . . can be selected for suitability in an as such known manner, taking the test's dynamic range into account. If, for example, a sample with a very high concentration and one with a zero concentration are available, by diluting them together, for example, 1:2 or 1:3, the next sample is obtained, which is further diluted, for example, correspondingly, etc. Genuine samples or completely artificial samples can also be used (e.g., purified proteins in a buffer). The sample's type is selected as reliable according to experience, i.e. sometimes a so-called sample matrix (such as blood serum or whole blood) can be included in the sample, so that the sample will act reliably. In some other product a purified analyte, diluted in a buffer, can be used.

As stage 1703, a group of samples with a known concentration C₀, C₁, C₂, . . . i.e. in this case the calibration samples S₀, S₁, S₂, . . . , are arranged for the group 49 of test devices 10′ selected from the test-device batch 29, and equipped with an indicator part 20 for measuring one or more analytes 30 from the sample, i.e. from the calibration samples S₀, S₁, S₂, . . . . In the case according to the described embodiment, the calibration sample S₀, S₁, S₂, . . . is put on the sample pads 43 of the test strips 10 (FIG. 16a).

Further, as stage 1703, the samples are measured, i.e. the calibration samples S₀, S₁, . . . , Sₙ using the measuring device 11 from one or more detection areas 21 arranged on the test device's 10′ indicator part 20 and equipped with test chemistry 26 for the analysis of the sample S₀, S₁, S₂, . . . In the measurement, the relevant detection area 21 of the test strip 10, i.e. in the example's case the test line 21′, is read, for example, at a specific set moment in time t₁, t₂, t₃, . . . . Alternatively, i.e. instead of the point-like reading moments in time, the measurement signal formed from the reading of the test line 21′, i.e. the development of the measurement variable 23, can also be monitored continuously, or at some preset (short) intervals, which also give results at the relevant moments in time t₁, t₂, t₃, . . . .

The reading interval can be dynamic, i.e. not necessarily constant, instead it can also change as time progresses. In other words, in any case, the measurement for each calibration sample S₁, S₂, . . . , Sₙ is made at several different moments in time. These moments in time, t₁, t₂, . . . , tₙ, thus form a time series, i.e. the question is of a time-series measurement, measured on at least one test strip 10 for the relevant one calibration sample S₁. As the calibration sample S₁ progresses on the test strip 10, the situation at the test line 21′ (the read measurement signal 23) changes as a function of time. Thus, at each moment in time t₁, t₂, . . . , tₙ, measurement signals M₁, M₂, . . . , Mₙ deviating from each other are obtained.

It is recorded at each moment in time t₁, t₂, . . . , tₙ belonging to the time series intended to be formed. It is characteristic for the measurement signal M₁, M₂, . . . that it increases as time progresses, because more stamped analyte collects on the test strip's 10 test line 21′ as time progresses. I.e., in other words, the measurement can be said to be increasing in measurement variable 23 i.e. as measurement signal 23. The vertical columns of FIG. 1 show the measurement signals at different measurement moments t₁, t₂, . . . tₙ for each different concentration C₀, C₁, C₂, . . . for the sample S₀, S₁, S₂, . . . .

At high concentrations, the measurement signal 23 as a function of time stops rising and can even drop, due to the aforesaid Hook effect. In addition, even smaller concentrations can reach the same signal level as time progresses. Thus, as time progresses the signals of samples with a lower concentration also rise to become greater, thus corresponding to the signal given by a higher concentration, especially in a situation in which the Hook effect acts. As a result of this, the signals of samples with a lower concentration can mix with the signals of samples with a higher concentration, so that a sample's real concentration can no longer be determined, if measurement continues for too long.

The same measurement series can be repeated using several test strips 10 for this sample S₁ with a single concentration C₁. From the measurement signals of these measurement series (for example, of five parallel measurements) a mean value can then be formed at each moment in time t₁, t₂, . . . , tₙ, which then form a time series for the relevant concentration C₁. In addition, in an as such known manner, the measurement can also be used to obtain data on the test strips' 10 deviation, while the quality of the test strips 10 can also be evaluated.

At different moments in time t₁, t₂, . . . , tₙ of the measurement, of the measurement signal (M₁, M₂, M₃, . . . )—concentration C₁ point pairs obtained by using the relevant calibration sample S₁ (i.e. the measurement signals') mean values, if several measurements have been made using a single concentrations' C₁ sample S₁ ((M₁, C₁), (M₂, C₁), (M₃, C₁), . . . ), corresponding response curves can be formed. A specific response curve can be formed for each moment in time t₁, t₂, . . . , tₙ of the time series. Because the sample's Si₁ concentration C₁ is known in each response curve of different moment in time t₁, t₂, . . . , tₙ belonging to the time series, the measurements can be represented as a point pairs of measurement signal Mx (Y-axis)—concentration Cx (X-axis). FIGS. 3, 4, and 23b show the response at different points in time for different concentrations. In turn, FIGS. 2 and 23a show all the development curves 38.2 formed from FIG. 1′s development data 38.1 for the different concentrations' C₀, C₁, . . . , Cₙ calibration samples S₀, S₁, . . . , Sₙ as time progresses. The question is now of the development of a lateral-flow assay at different concentrations C₀, C₁, . . . , Cₙ.

Time-series measurements (FIG. 1′s vertical columns) are performed and the point pairs (Mₓ, Cₓ) obtained on their basis are taken to the set of co-ordinates corresponding to the moments in time t₁, t₂, t₃, . . . also for the other calibration samples S₂, S₃, . . . , Cₙ, i.e. the time-series measurements and formation of response curves at different concentrations C₂, C₃, . . . . Thus, as a result, at every moment in time t₁, t₂, t₃, . . . in the corresponding response curve there are measurement results from several different measurements and thus also from different test strips, in which in addition the calibration samples' S₀, S₁, S₂, . . . concentrations C₀C₁, C₂, . . . deviate from each other. Thus, the measurement signal's 23 development as a function of time using different test strips 10 and the calibration samples S₀, S₁, S₂, . . . with different concentrations C₀, C₁, C₂, . . . is obtained. Once again reference is made to FIGS. 3, 4, and 23b, and in addition to FIGS. 2 and 23a (relating to FIG. 1).

Thus, concerning the aforementioned stages, one can speak of the formation of response curves using measurements performed at different points in time on different concentrations. In other words, in connection with the development of the measurement of each sample, at least some of the calibration samples S₀, S₁, . . . , Sₙ are measured on the detection area 21, to form development data 38.1 for the calibration sample's S₀, S₁, S₂, . . . various concentrations C₀, C₁, C₂, . . . . Based on the measurements, development curves corresponding to different concentrations can be formed.

The problematiques relating to measurement can also be explained with the aid of FIGS. 1 and 3, to which the invention particularly applies. The largest measurement signal of each measurement is circled in FIG. 1′s concentration columns and in FIG. 3 time columns. It can be seen particularly from the measurements of the samples' Sₙ, Sₙ₋₁ of very high concentrations Cₙ, Cₙ₋₁, that in them the greatest measurement signal is not obtained from the measurement of the largest sample concentration Cₙ, but already earlier at the concentration Cₙ₋₁. Thus, the measurement signal 23 may even drop at the highest measured concentration Cₙ. These decreasing measurement signals are circled by broken lines in FIGS. 1 and 3. The same is also seen from FIG. 4′s response curves, especially in the measurement of time point t₂, i.e. 5.08 min. This phenomenon taking place at high concentrations is known as the Hook effect. As a result of it, erroneous measurement results are obtained, especially at high sample concentrations.

FIGS. 5, 6, and 23c show, for example, data corresponding to two different points in time t₁ (1.13 min) and t₂ (5.08 min), and the corresponding response curves formed from them. Again it can be seen from these that in the longer measurement lasting to point in time t₂ the measurement signal 23 at the highest concentration (3486 mg/l) of sample Sₙ is lower than the measurement signal of the sample Sₙ₋₁ (1743 mg/l) with the preceding concentration.

As stage 1705, calibration data 25.1 is formed on the basis of the development data 38.1 for at least one moment in time t₁, t₂ in connection with the development of sample's 19 measurement, to form a calibration function 32.1, 32.2 for the relevant measurement at at least one moment in time t₁, t₂. In other words, calibration functions 32.1, 32.2 are formed on the basis of the development data for different moments in time. This can be done, for example, in such a way that a function adaptation is made for the point pairs formed on the basis of the measurements performed above in the response curves at different moments in time t₁, t₂, t₃, . . . . I.e. in the response curve of moment in time t₁ an adaptation is made for the point pairs (M₁, C₀; M₁, C₁; M1, C₂; M1, C₃; M1, C₄; and M1, C₅), in the response curve of moment in time t₂ for the point pairs (M₂, C₀; M₂, C₁; M₂, C₂; M₂, C₃; M₂, C₄ ja M₂, C₅), etc. to form corresponding calibration curves to each response curve corresponding to each moment in time t₁, t₂, t₃, . . . of each time series. Even more generally, one can speak of the formation from the calibration curves of a function dependent on the development time. Hill's function is an example of this connection. Instead of the function adaptation, a time point calibration curve of one or more points in time can also be used, which can also be understood in this context as a calibration function. In addition, one or more of the calibration functions 32.1, 32.2 formed, the derivative of which is positive, is selected and recorded. The calibration function's 32.1, 32.2 derivative can be said to be positive, if its curve rises.

FIG. 7 and the corresponding FIGS. 8 and 23d show the data relating to this stage, i.e. calibration at different points in time. Each time point can have its own calibration function 32.1, 32.2. It is fitted to the measured data from the response at the relevant point in time. FIGS. 9, 10, and 23e show, for example, the data corresponding to two different point in time t₁ and t₂ and the calibration curves formed from them. Now, as the question is of a function adaptation, a calculated measurement signal can be shown also at high concentrations, if the measurement would proceed according to the calibration adaptation.

FIG. 17b shows in greater detail one embodiment in the case of the stages following calibration, as a continuation of stage 1705.1, which was touched on earlier in connection with FIG. 17a, as stage 1705. As stage 1705.2, the calibration curves 32.1, 32.2, corresponding to different points in time, and which is formed, for example, by adapting the function to each point group of the calibration curve, is analysed. In the analysis, one or more ranges of a function, the derivative of which is continually positive, can be defined from each calibration curve 32.1, 32.2, i.e. from each point in time corresponding to them. In addition to the above, in the analysis, as stage 1705.3, it is also defined that in the value ranges of the function with a continually positive derivative, their derivative has also a sufficient value for the target resolution to be achieved, taking the tests' measured deviation into account. In other words, one can speak of the definition of the quantification range of the relevant moment in time.

Further, each test-device manufacturer can, for example, define its own resolution criteria for its own products, entirely independently. Here, for example, the deviation (SD—standard deviation) can be calculated at the relevant moment in time for the parallel measurements of each sample and examine (e.g. visually or by calculating approximations) or by setting directly in the function the resolution at any point—i.e. the smallest step in the rise in concentration that can be reliably observed, taking the tests' deviation into account. Typically the criterion used is deviation (SD) or its multiple, for example 3×SD, which is already a very tight criterion. Alternatively, the share of deviation of the signal, i.e. the so-called CV (coefficient of variation) can also be calculated. This expresses the variation as a percentage of the signal. To give one example, the target can be, for example, <10% CV over the whole quantification range.

The calibration curve (or part of it) can also be, for example, a straight line, with a set kind of slope. Instead of straight line, to the point group can, using a calculation program (e.g. MS Excel) also fit some other kind of curve, so that straight line is not the only possibility. One or more parts formed from the calibration curve is, however, continuously rising with sufficient steepness (angle coefficient) for it to have an acceptable resolution and, in addition, the correspondence of the measurement signal—concentration should be continuous. In other words, the relevant part of the curve should indicate an increasing measurement signal, and thus also, as a result, an increasing concentration. In other words, the measurement signal should (in resolution clearly) increase as a function of concentration C at the relevant selected moment in time t₁, t₂, t₃, . . . . This condition should be realized in each calibration curve 32.1, 32.2 selected to the calibration 25 of the measuring device 11. Thus, this operation too (adaptation and search for curve areas meeting the criteria) is done at several moments in time t₁, t₂, t₃, . . . , of which one or more suitable moments in time are then selected for the calibration 25 formed for the measurement of an unknown sample 19.

As stage 1706 of FIGS. 17a and 17b, a set of criteria Kₜ₁, Kₜ₂ are formed for the measurement of sample 19, such as, for example, for determining the usability of sample's 19 measurement and, in addition, for controlling sample's 19 measurement. The set of criteria Kₜ₁, Kₜ₂ is formed by defining at least one criterion value H or one or more criterion values H, L from each of the selected calibration functions 32.1, 32.2 with a continually positive derivative and also sufficient resolution, which are included in the measurement device's 11 calibration 25.

As the criterion values H, L linked to each calibration functions 32.1, 32.2 are defined at least one of the following: at least one measurement area's upper limit H or data corresponding to it, or at least one measurement area's upper limit H and lower limit L or data corresponding to these. The criterion values H, L are recorded, for example, with the calibration data 25.1, i.e., for example, with the calibration parameters. On the basis of the calibration parameters, i.e. the calibration data 25.1, calibration curves 32.1, 32.2 can be formed on the measuring device 11, in connection with which the criterion values H, L, belonging to the set of criteria Kₜ₁, Kₜ₂, are applied. The upper and lower limits H, L define the usable measurement area of the calibration function 32.1, 32.2, i.e. now the usable measurement area's upper limit H and the usable measurement area's lower limit L.

As stage 1706.2, in FIG. 17c categorization for the measurement of the sample 19 is formed on the basis of the criterion values H, L belonging to the set of criteria Kₜ₁, Kₜ₂. The categorization now also acts as an operating instruction for the measuring device 11 and the measurement of the sample 19 being performed by it. The categorization and thus also the operating instruction includes one or more of the following. As stage 1706.3, in connection with the development of the measurement of the sample 19 by the measuring device 11 it is set to be defined whether the measurement result 23 exceeds the criterion value H set for it. This criterion value H defining the measurement range's upper limit can also be called a primary criterion value. If the measurement variable 23 defined from the sample 19 being measured at the relevant moment in time t₁ is above the set criterion value, which is now the upper limit H of the usability of the measurement range defined in connection with calibration, measurement of the sample 19 is set to be ended on the measuring device 11 at the moment in time t₁ defined in the relevant calibration 25. Thus, the measurement is shown to be unusable, as the measurement result 23 of the sample 19 is not in the usable measurement range defined from the calibration function 32.1. The measuring device's 11 operator can then also be requested, for example, to dilute the sample 19 being analysed and to start from the beginning the measurement of the sample 19 to be analysed and perform it again. In other words, in stage 1706.2, is defined and set for the measuring device 11 on the basis of the criterion value H belonging to the set of criteria at least one or more concentration ranges (H, Hv) from the calibration function 32.1, 32.2 of at least one moment in time t₁, t₂, by which the measurement is set to be unusable by the measuring device 11, for the concentrations of the sample 19 defined by the range (H, Hv) in question.

If the measurement variable 23 measured from the sample 19 measured at the relevant moment in time t₁ is less than the set criterion value, which is the measurement area's upper limit H, the sample's 19 measurement is categorized as usable, at least at this moment in time t₁ defined by the relevant calibration 25, when the relevant measurement variable 23 was defined from the relevant sample 19 being measured. This is noted as stage 1706.4. In other words, here from the selected calibration function 32.1, 32.2 one or more concentration ranges (Lv, L), [L, H] are defined, by which a measurement, performed before the end point of the measuring procedure of the test device 10′ set for the measuring device 11, is usable at the concentrations of the sample 19 defined by the range in question.

As stage 1706.5, the measuring device 11 is set to define whether there is, in the calibration 25 arranged for it, yet another criterion value for the same relevant moment in time t₁. In the embodiment according to the example, it is now the measurement range's lower limit L in the calibration function 32.1 at the relevant moment in time t₁. If this has not been set, as stage 1706.7, the measurement of the sample 19 is set to be continued to the following moment in time t₂, defined in the calibration 25. If in stage 1706.5 it is found that it has been set, then move to stage 1706.6. There it is examined whether the measurement variable 23 measured from the sample at the relevant moment in time t₁ is in the measurement range between the lower limit L and the upper limit H bound to the calibration function 32.1. If it is, as stage 1706.8, the analysis result 24 is set to be defined and reported.

On the other hand, if as stage 1706.6 it is found that the measured measurement variable 23 is not at the relevant moment in time t₁ in the range between the criterion values, i.e. now between the upper limit H and the lower limit L of the usable measurement range of the calibration function 32.1, in other words that it is below the set lower limit L, then as stage 1706.7 the sample's 19 measurement is set to be continued at the following moment in time t₂ defined by the calibration 25.

The same is done for each selected measurement moment in time t₂ belonging to the calibration 25 and to the calibration functions 32.2 corresponding to them. In other words, for each moment in time t₁, t₂, t₃, . . . a measurement signal's lower limit L (Lₜ₁, Lₜ₂, Lₜ₃, . . .) and a measurement signal's (and thus also a concentration's) upper limit H (Hₜ₁, Hₜ₂, Hₜ₃, . . .) is defined on the basis of the curve portions meeting the set criteria defined above. Again more generally, one or more sub-areas of the function 32.1, 32.2 fitted to the point group is defined in each point in time t₁, t₂, t₃, . . . of the sample's 19 measurement defined in the calibration 25, the derivative of which is continuously positive and in addition in value with sufficient resolution in the aforementioned manner. These limits L, H act as usability criteria of the curves 32.1, 32.2 and thus also of the measurement in each calibration curve 32.1, 32.2, i.e., at each point in time t₁, t₂, t₃, . . . of calibration 25 in the measurement of a test sample 19 with an unknown concentration, at different points in time of the measurement, of which some are in connection with the development of the sample's 19 measurement on the test device 10′. In other words, the upper and lower limits H, L tell of the concentration range of the sample being measured, by which the calibration curve 32.1 formed at the relevant moment in time t₁ can be applied in the measurement at the relevant moment in time t₁.

Thus, on the basis of the measurements, test-device batch's 29 calibration 25 (calibration data) can be said to be formed for the sample 19 to be analysed. Calibration data 25.1 concerning one or more detection areas 21 can be said to belong to the calibration 25. On the basis of the calibration data 25.1, such as, for example, the calibration curve 32.1 formed of that for at least one moment in time t₁, is information 28 concerning the sample 25 with an unknown concentration, to be formed by a measuring device 11 equipped with calibration 25, such as, for example, a numerical analysis result 24 of the sample 19. In addition, the calibration 25 can be said to also include the aforementioned set of criteria Kₜ₁, Kₜ₂, the criterion values L, H (and the ranges defined by them ((Lv, L), [L, H], (H, Hv)) belonging to it, the categorization formed on their basis, and the measuring device's 11 operating instruction based on the categorization, being based on the measurement's usability data for controlling the progress of the sample's 19 measurement, and/or to form information 28 concerning the sample's 19 measurement.

As stage 1707, the calibration data 25.1 concerning the test-device batch 29 is recorded in a data structure 18 formed for calibration 25, for taking to the measuring device 11. In addition as stage 1707 the set of criteria Kₜ₁, Kₜ₂ belonging to the calibration 25, together with the criterion values L, H and a possible corresponding categorization of the measurement of the sample 19, together with operating instructions, are also recorded. The categorization with the operating instructions can also be included in the computer program 37′, i.e. for example in the measurement program 37, which is executed using the measuring device 11 during the performance of the measurement of the sample 19.

Thus, the invention also concerns a data structure 18 for the calibration of the measuring device 11. FIG. 9 shows very schematically a possible example of the content of such a data structure 18. The data structure 18 includes as a calibration 25 calibration data 25.1 for forming one or more calibration functions 32.1, 32.2 for the measuring device 11 for the measurement of the sample 19 at at least one moment in time t₁, t₂. Thus, the calibration function 32.1, 32.2 can be said to be adapted to correspond to the moment in time t₁, t₂ set in the measuring device 11 for performing the measurement of the test device 10′ for measuring the sample 19. At least one moment in time t₁ of the moments in time t₁, t₂ of the measurement of the sample 19 is in connection with the development of the sample's 19 measurement.

In addition, the calibration 25 arranged in the data structure 18 also further includes a set of criteria Kₜ₁, Kₜ₂. The set of criteria Kₜ₁, Kₜ₂ is for defining the usability of the sample's 19 measurement at at least one moment in time t₁. At least one of the moments in time t₁ is in connection with the development of the sample's 19 measurement. In addition to the definition of usability, the set of criteria Kₜ₁, Kₜ₂ is also for controlling the progress of the sample 19′s measurement and/or for forming information 28 concerning the sample 19 at one or more moments in time t₁, t₂ corresponding to the relevant one or more calibration functions 32.1, 32.2. As described in the example above, the data structure 18 can have been formed in connection with the factory calibration of the test-device batch 29.

As stage 1708, a data-structure record 34 is formed by arranging a data-structure 18 to a data-carrier 39 to take the calibration to the measuring device 11. FIG. 19 shows a rough schematic diagram as an example of the data-structure record 34. Thus, the invention also equally relates to a data-structure record 34, which includes a data structure 18 according to the invention described, such as, for example, in FIG. 9, and its carrier 39. The carrier 39 can be, for example, a storage device equipped with a memory 15, such as, for example, a memory card, which is often also called a calibration card 34′. The data structure 18 is arranged in the memory 15 of the carrier 39 to take the data-structure 18 to the measuring device 11 and for the calibration of the measuring device 11 for the measurement of the sample 19 at one or more moments in time t₁, t₂. At least one moment in time t₁ of the moments in time t₁, t₂ is in connection with the development of the sample's 19 measurement, i.e. before the end point of the test device's 10′ measurement possibly preset for the measuring device 11. Using the data-structure record 34, such as, for example, a calibration card 34′, the calibration parameters (or similar data) formed according to the method can be transferred to the measuring device 11 for performing the measurement method according to the invention. For this, the calibration card 34′ can have data-transfer interface 40, for which the measuring device 11 has its own data-transfer interface 41.

As stage 1709, a test-device series 31 is formed of the test devices 10 of the test-device batch 29, and thus also of those that are the objects of the calibration process described above, and the data carrier 39 containing the calibration 25. This can be, for example, a group of twenty test strips 10 and a calibration card 34′. The test strips 10 can have, for example, a 12 or 18-month usability period.

Thus, the invention also relates to the actual test-device series 31, of which an example is shown in FIG. 20. The test-device series 31 includes a test-device group 49′ formed from a test-device batch 29, and a data structure 18 for taking the calibration 25 concerning the test devices 10′ to the measuring device 11 for measuring the sample 19 by the test device 10′. The devices 10′ are equipped with an indicator part 20. The indicator part 20 includes in turn one or more detection areas 21. The detection areas 21 are equipped with test chemistry 26 for measuring the sample 19. The test devices 10′ are calibrated using the method according to the invention.

The test-device series' 31 data structure 18 is, in turn, arranged on the data carrier 39 to take the calibration 25 concerning the test devices 10′ to the measuring device 11 to measure the sample 19 using the device 10′. The calibration 25 includes calibration data 25.1 concerning the test devices 10′. The calibration data 25.1 is for forming information 28 concerning the sample 19. In addition, calibration 25 includes the set of criteria Kₜ₁, Kₜ₂ already described above, together with the criterion values and the categories they define. In other words, the data structure 18′ is a data structure 18 according to the invention.

FIG. 11 shows the values of the response measurements of the time points converted with the aid of the time-point specific calibration function into the concentration of the sample, while FIGS. 12 and 23f FIG. 11 corresponding curves. One can also speak of the linearity at different points in time. FIG. 13 shows the data of FIG. 11 at two different points in time t₁ and t₂ and FIGS. 14 and 23g a corresponding curve at the different points in time t₁ and t₂. Using the data it can, according to the aforementioned principle, be defined (and seen visually from the curve), that, for example, the usability of a measurement performed at time point t₁ (the measurements' deviation meets the set criterion, being less than it) is in the concentration range 80-2000 mg/l. In other words, the lower limit Lₜ₁ at the moment in time t₂ (1.13 min) of the measurement's usability can be set as 80 mg/l and the measurement's usability upper limit Hₜ₁ at the moment in time t₁ as 2000 mg/l. These limits Lₜ₁, Hₜ₁ are applied later in measuring a test sample of unknown concentration with the relevant calibration curve at the relevant moment in time t₁. It should be noted that the example and the numerical values given above were at this point completely principled and visually defined from the curve.

Similarly, using the data it can be determined using the aforementioned principle that the usability of a measurement performed (the deviation of the measurements meet the set criterion, being less than it), for example, at a moment of time t₂ is in the concentration range 20-1000 mg/l. From the data and the curve too the influence of the Hook effect on the measurement can be clearly seen at concentrations of more than 1000 mg/l, especially at the final concentration (3486 mg/l) of the measurement series. In other words, once again the lower limit Lₜ₂ of the usability of the measurement at the moment in time t₂ (5.08 min) can be set as 20 mg/l and the upper limit Hₜ₂ of the usability of the measurement at moment in time t₂ as 1000 mg/l. These limits Lₜ₂, Hₜ₂ are applied later by the measuring device 11 in measuring a test sample with an unknown concentration with the relevant calibration curve at the relevant moment in time t₂.

FIG. 18a shows an example of the method according to the invention as of the measuring device's 11 measuring procedure, according to one embodiment, for measuring a test sample with an unknown concentration, i.e. more generally a sample 19. FIG. 18b in turn shows a similar measuring procedure to FIG. 18a, but on a slightly more general level. In addition, the flow diagram of FIG. 18b further relates to a situation, in which the calibration 25 of the test-device series 31 has already been completed for the measuring device 11.

As stage 1800 of FIG. 18a, the calibration 25 of the test device 10′ to be used in the measurement of the sample 19, which test device 10′ belongs to the test-device batch 29, which the calibration in question concerns is arranged for the measuring device 11. This can be done, for example, using a data-structure record 39, the formation of which was described slightly earlier in connection with the description of the calibration method. Using it, the data structure 18 formed in connection with the test-device batch's 29 calibration together with the data t₁, t₂, 32.1, 32.2, Kₜ₁, Kₜ₂, H, L stored in it is downloaded to the measuring device 11 for measuring the sample 19 using a test device 10′ belonging to the test-device series 31 formed from the test-device batch 29.

Stage 1800 can be divided, for example, into two sub-stages 1800.1, 1800.2. As sub-stage 1800.1, calibration data 25.1 for forming one or more calibration functions 32.1, 32.2 on the measuring device 11 for the measurement of sample 19 is downloaded and stored, more generally, arranged, on the measuring device 11. At least one of the calibration data 25.1 and thus also of the calibration functions 32.1, 32.2 formed from them, concerns the moment in time t₁ in connection with the development of the measurement of sample 19. The measurement variable 23 measured from the sample 19 by the measuring device 11 can then also be said to be rising in value at the relevant moment in time t₁.

As sub-stage 1800.2 in turn, a set of criteria Kₜ₁, Kₜ₂ relating to one or more calibration functions 32.1, 32.2 is downloaded and stored, more generally, arranged, in the measuring device 11. The set of criteria Kₜ₁, Kₜ₂ is for the definition of the usability of the measurement of sample 19 in connection with the development of the measurement of the sample 19 by the measuring device 11, and in addition, the progress of the measurement of the sample 19 and thus also for controlling the operation of the measuring device 11 on the basis of the measurement of the sample 19.

Thus, stage 1800 can be said to be the input to the measuring device 11 of the selected calibration curves 32.1, 32.2 (or the corresponding data required to form them, i.e., for example, the calibration parameters) of the formed calibration curves 32.1, 32.2, together with their usability data and their related upper and possible lower limits H, L at different moments in time t₁, t₂, along with the corresponding operating instructions, which can also be part of the sets of criteria Kₜ₁, Kₜ₂. In other words, factory calibration formed beforehand for the test-device batch 29 is then set for the measuring device 11. Then, in the measurement of the sample 19, the measuring device 11 should use the test-device group 49′ formed from this relevant test-device batch 29, for which the calibration in question has been made in connection with the development of the measurement of the relevant sample 19, which it thus now concerns. In other words, the calibration 25 with the sets of criteria Kₜ₁, Kₜ₂ and calibration data 25.1 can be said to be arranged for the measuring device 11 in the form of the factory calibration of the manufacturing batch of test devices 10′.

With reference to FIG. 18a and now also 18b, as stage 1801 the sample 19 to be analysed, i.e. the test sample, is arranged on the test device 10′, i.e. in the embodiment described on the test strip 10 (-base etc.) for the measurement of the sample 19. The test device 10′ has already been equipped as described above with an indicator part 20 for measuring one or more analytes 30 from the sample 19.

As stage 1802, the sample 19 to be analysed is measured by the measuring device 11 from one or more detection areas 21 arranged on the test device's 10′ indicator part 20 and equipped with test chemistry 26 for the analysis of the sample 19. Thus, the test device 10′, such as, for example, a test strip 10 is placed in the measuring device 11 and the measurement of the test device 10′ with the sample 19 is started. In the measurement, a measurement signal is formed from the test device 10′, i.e. now from one or more detection areas 21 of its indicator part 20. The measurement signal can also be called the measurement variable 23 or measurement result. Thus, it is, for example, an electrical primary variable, which the measuring device 11 forms from the test device 10′ in the measurement in connection with the performed measurement of the sample 19.

In stage 1802, the measuring device 11 is used to measure one or more detection areas 21, i.e. the sample 19 to be analysed, at the moment in time t₁ in connection with the development of the sample's 19 measurement. The measurement of the sample 19 can be said to develop in the test device's 10′ detection area 21, because the measurement signal formed in the sample's 19 measurement, i.e. the measurement variable 23, changes as a function of time. Thus, every measurement moment of time, at which the sample 19 is measured to form a measurement result 23 from it and to analyse it, also requires its own calibration (curve), because the measurement signal increases, i.e. more generally changes, continuously as the sample's 19 measurement develops in the test device 10′. One can also say that the measurement is then performed at at least one moment in time t₁, which is before the end point of the measurement of the test device 10′ typically preset on the measuring device 11. Measurement not until the measurement's end point of the test device 10′ set for the measuring device 11 is a known form of operation in known measurement methods for performing the measurement of a sample 19.

Thus, in stage 1802, one can say that the sample 19 is allowed to develop in the test device's 10′ indicator part 20, and now even more particularly, its detection area 21, when it is measured using the measuring device 11. Here the development of the sample 19 means, for example, in the case of a lateral-flow assay 10*, for example, to at least one or more of the following: the sample's 19 progression on the test strip 10, the sample's 19 reaction with the test chemistry 26 arranged on the test strip 10 and/or particularly the stamped sample's 19 accumulation in one or more detection areas 21 of the indicator part 20 for the reading of the test, i.e. the measurement of the measurement variable 23 taking place in the measuring device 11. From these, especially from the last of them it can also be said that the measurement variable 23 is measured in connection with the sample's 19 development at least at one moment in time t₁, when the measurement variable 23 measured from the sample 19 has a value that is an increasing as function of time.

In stage 1802, the test line 21′ of the test strip 10 is measured at at least one moment in time t₁ defined in the calibration 25 and brought to the measuring device 11. The moment in time t₁ is defined from the time series measurement performed in connection with the calibration measurements of the test-device series 31 and from the calibration functions 32.1 formed on their basis. As already noted earlier in the method description of the calibration measurement, it is not necessarily possible to provide any generally applicable criterion for the sample's 19 measurement moment in time t₁. However, it is generally possible to estimate, for example, the steepness of the rise of the measurement signal defined in the test-device series 31 calibration stage and the deviation of the measurement results 23 in calibration and on their basis to define one or more calibration functions 32.1, 32.2 and thus also the moment in time t₁, t₂,, that suits the measurement's performance in the way according to the invention.

On the other hand, though the measurement results' deviation would be great at the first moment in time t₁ at which the measurement is performed, the samples 19 with already very high concentrations, for example, can nevertheless be identified at the relevant moment in time t₁. This is especially so if, for example, the steepness of the calibration curve 32.1 is sufficient at the relevant moment in time t₁. In the factory calibration of the test-device batch 29, the measurement's first moment in time t₁ is set to be some specific moment in time, i.e. when using the product (a test strip 10 belonging to the relevant test-device batch 29), all the test-device's 10′ measurement moments in time, and the measurement results' upper and lower limits H, L, by which the measuring device's 11 operation and the progress of the measurement are defined, are known precisely beforehand in the measuring device 11, which then performs the sample's 19 measurement according to them.

The moment in time t₁, when the first measurement is performed and on its basis the first measurement indication is also given to the end user of the measuring device 11, can be (even considerably) earlier than the measurement time defined and set for the measurement (for the test device 10′), i.e. the end point of the measurement procedure set for the test device 10′, at which the measurement result is formed and reported according to the prior art. One exemplary criterion for this preset measurement's end point can be, for example, that then the test strip 10 can be entirely wet with the sample 19.

As stage 1803, the usability of the sample's 19 measurement is defined on the basis of the measurement performed and of the calibration 25 of the test-device batch 29 arranged for the measuring device 11 and of the set of criteria. I.e., this too takes place in connection with the development of the sample's 19 measurement, at the moment in time t₁ defined in test-device batch's 29 calibration. In other words, this too can be said to take place, at least once, before the end point of the test device's 10′ measurement procedure set for the measuring device 11.

On the basis of the definition of the measurement's usability, the progress of the sample's 19 measurement can be controlled on the measuring device 11. In addition, on the basis of the definition of the usability of the sample's 19 measurement, information 28 can also be formed concerning the sample 19 or even more generally, the sample's 19 measurement.

The definition of the measurement's usability and in addition the control of the progression of the measurement are based on the set of criteria Kₜ₁ belonging to the calibration 25 and formed in connection with the development of the sample's 19 measurement at at least one moment in time t₁. The set of criteria Kₜ₁ in turn includes at least one criterion value H defined from the calibration function 32.1 corresponding to the relevant moment in time t₁. On the basis of the criterion value H, the measurement to be performed on the sample 19 can be categorized at the relevant moment in time t₁ into two categories A, BC. The categorization now relates to the usability of the measurement. The categorization is based on the value ranges ((Lv-L), [L-H] (H-Hv)) defined from the calibration function 32.1 on the basis of one or more criterion values H, L at the relevant moment in time t₁, t₂.

If, only one criterion value H, i.e. a criterion value H defining the upper limit of the usable measurement area of the calibration function 32.1, 32.2 is formed in calibration, then the value areas can be, for example, (Lv-H], (H-Hv). If, in turn, in calibration two criterion values H, L are formed at the relevant moment in time t₁, i.e. the criterion value H defining the upper limit of the usability of the calibration function 32.1 and, in addition, the criterion value L defining the lower limit of the usability of the calibration function 32.1, the value ranges can be (Lv-L], [L-H], and (H-Hv). In both, Lv and Hv refer to the starting point and end point of the calibration function. On the basis of those value ranges (Lv-L], [L-H], and (H-Hv), the usability of the sample's 19 measurement is defined in connection with the development of the sample's 19, and/or the progress of the measurement in connection with the development of the sample's 19 measurement is controlled, at the stages described in greater detail in the following.

In stage 1803.1 shown in greater detail in FIG. 18c, the measurement result 23 of the sample 19 formed by the measuring device 11 at the relevant moment in time t₁ is analysed. If it is found in stage 1803.1 that at the relevant moment in time t₁ the measurement signal 23 measured from the sample 19 on the detection area 21 is greater than the upper limit H at the relevant moment in time t₁ defined from the calibration curve 32.1, the progress of the measurement is controlled so that the sample's 19 measurement is interrupted.

As stage 1803.2, the measuring device's 11 operator can be notified, as information 28 concerning the sample 19, that the test sample's 19 concentration exceeds the test's dynamic range and the measurement has been interrupted. In addition, the operator can be instructed, for example, to dilute the sample 19 and perform the measurement again. Thus, on the basis of the set of criteria Kₜ₁, Kₜ₂, and even more particularly of at least one criterion value H belonging to it, the sample's 19 measurement is rejected, if the measurement variable 23 formed using the measuring device 11 on the basis of the measurement of the detection area 21 exceeds the criterion value H belonging to the set of criteria Kₜ₁ at the corresponding moment in time t₁. In other words, the sample's 19 measurement result is then in connection with the development of the measurement of the sample 19 in at least one value range (H-Hv)) defined from the calibration function 32.1 on the basis of the criterion value H and on its basis the progress of the sample's 19 measurement is controlled in such a way that the sample's 19 measurement is interrupted.

If, on the other hand, it is shown in stage 1803.1 that at the relevant moment in time t₁ defined by the calibration 25 the measurement signal 23 measured from the sample 19 in the detection area 21 is less than the upper limit H of the usable measurement area defined from the calibration curve 32.1 at the relevant moment in time t₁, a move is made to stage 1804. In stage 1804, the sample's 19 measurement is continued, because as stage 1803.1 the sample's 19 measurement was found to be, at least at the relevant moment in time t₁, still usable. Thus, the calibration 25 then includes the set of criteria Kₜ₁ with the criterion value H, formed at least at one moment in time t₁.

In other words, in the case according to the embodiment, the categorization A, BC made of the basis of the value ranges (Lv-H], (H-Hv) defined by at least one criterion value H belonging to the set of criteria Kₜ₁, Kₜ₂, concerning the usability of the sample's 19 measurement and/or the control of the progress of the measurement in connection with the development of the sample 19 defined at the moment in time t₁, can comprise one or more of the following: at least one value range (H-Hv) of the measurement result 23 defined in stage 1803.1, by which the sample's 19 measurement is rejected (usability category A) and at least one value range (Lv-H] of the measurement result 23, on the basis of which the sample 19 is measured to define the analysis result 24 from the sample 19 as stage 1804 (as usability category BC). Thus, the categorization can be said to be based on the measurement result's 23 value ranges (Lv-L), [L-H], (H-Hv) defined from the calibration function 32.1, 32.2 on the basis of the criterion values L, H.

As stage 1804, the progress of the measurement is controlled and/or information 28 concerning the measurement of the sample 19 is formed on the basis of the usability of the measurement found in stage 1803.1. If the criterion values were only one at the relevant moment in time t₁ in connection with the development of the sample's 19 measurement, i.e. the upper limit H already described in the aforementioned stage 1803.1, then the measurement result of the sample 19 has to be in the value range defined by the criterion value H of the set of criteria Kₜ₁ in stage 1804.1, i.e. in the calibration function's 32.1 value range (Lv-H), which is thus now the value range of the calibration function 32.1 below the criterion value H. One then moves to stage 1804.4.

In stage 1804.2, the progress of the sample's 19 measurement is controlled in such a way that the sample's 19 measurement is continued to the next moment in time t₂ defined by the calibration 25, as set in the diagram of FIG. 17c.

Instead of that, if there were at least two criterion values at the relevant moment in time t₁, i.e. the already aforementioned upper limit H defined from the calibration function 32.1 at the relevant moment in time t₁, and in addition to that the lower limit L defined from the calibration function 32.1 at the relevant moment in time t₁, then in stage 1804.1 it is determined whether the sample's 19 measurement result is in the value range defined by the set of criteria Kₜu i.e. now in the value range L-H of the calibration function 32.1 at the relevant measurement moment t₁. It is characteristic of the one or more value ranges L-H defined by one or more criterion values L, H belonging to the set of criteria Kₜ₁, Kₜ₁, that in them the derivative of the calibration function 32.1 formed on the basis of the calibration data 25.1 is continuously positive. In addition, it is characteristic of the one or more value ranges L, H defined by the one or more criterion values L, H belonging to the set of criteria Kₜ₁, Kₜ₁, that the calibration function 32.1 formed on the basis of the calibration data 25.1 has sufficient resolution, taking the test devices' 10′ measured deviation into account. If the measurement variable 23, i.e. result, formed by the measuring device 11 on the basis of the measurement of the detection area 21 is in the value range [L-H] of one or more calibration functions 32.1 defined by the set of criteria Kₜ₁ corresponding this relevant moment in time t₁, more particularly its criterion value H, L, then too one progresses to stage 1804.2 already described above.

In stage 1804.2, the progress of the sample's 19 measurement is controlled in such a way that, of the basis of the set of criteria Kₜ₁, Kₜ₂, the sample's 19 measurement at the relevant moment in time t₁ is accepted and as the next stage 1805 is formed and reported as information 28 concerning the sample 19, the sample's 19, for example, numerical analysis result 24.

Thus, if the measurement signal is between the lower limit L and the upper limit H, defined from the calibration function 32.1 at the relevant moment in time t₁, the analysis result 24 is read from the calibration curve (FIGS. 10 and 23e) at the moment in time t₁. If, for example, the measurement signal at the moment in time t₁ were to be, for example, 4000, then the relevant sample's 19 final analysis result 24 would then, according to the calibration curve 32.1, be 1000 mg/l.

In the embodiment described of two criterion values H, L, if in stage 1804.1 it is found, on the basis of the measurement of the detection area 21, that the measurement variable 23, measured and formed by the measuring device 21, is less than the criterion value L defined from the calibration function 32.1 belonging to the set of criteria Kₜ₁, i.e. it is thus in the calibration function's 32.1 value range (Lv-L) and not in the value range defined by the set of criteria Kₜ₁, Kₜ₂, i.e. [L-H], the measurement result is then below the criterion value L. Then, on the basis of the set of criteria, and even more particularly of the criterion values L, H belonging to it, one moves to stage 1804.4. According to it, the sample's 19 measurement is continued to the following moment in time t₂ defined by the calibration 25. One then returns to stage 1802. Thus, the progress of the sample's 19 measurement and the operation of the measuring device 11 is once again controlled on the basis of the set of criteria Kₜ₁, Kₜ₂. The continuation of the measurement to the following time point t₂ can also be notified to the measuring device's 11 operator. In other words, at this point too information 28 can be formed concerning the sample's 19 measurement.

In other words, if it is found in stages 1804.2 and 1804.3 that the measurement signal 23 is less than the lower limit L defined from the calibration curve 32.1 of the relevant moment in time t₁, the measurement is allowed to continue, for example, to the end, or else to next one or more (now one) moments in time t₂ defined in connection with the calibration, i.e., for example, at the 5-minute point. Thus, the second, or more generally the last measuring time can be, for example, the measuring time (the ‘end’/development point of the measurement) set as the end point of the test device 10′ in question. It can vary, for example, depending on the test. Of course there can be still more moments in time between the first measurement and the end point preset for the performance of the measurement, by which the measurement can be performed according to the principle of the invention.

Thus, in the case of the two criterion values H, L, the calibration 25 and the set of criteria Kₜ₁, Kₜ₂ belonging to it can have been formed for at least two moments in time t₁, t₂. At least one of the moments in time t₁ is then in connection with the development of the sample's 19 measurement, i.e. before the end point set for the measuring procedure, defined from the calibration function 32.1 corresponding to the relevant moment in time t₁. The corresponding categorization, i.e. the operational control of the measuring device 11 then comprises three categories A-C. These are the measurement's interruption (A) in the value area of the calibration function 32.1, 32.2 exceeding the criterion value H, the formation and reporting of the analysis result 24 from the sample 19 (B) in the value area of the calibration function 32.1-32.2 between the criterion values H and L, and the continuation to the next point in time of the measurement (C) in the value range of the calibration function 32.1, 32.2 that is less than the criterion value L.

Above, the usability data of the measurement and, in addition, also the analysis result were obtained according to the invention already when the development of the sample's 19 measurement was in progress. The same result would be obtained equally if one waited for the end point set for the test, i.e., for example, up to 5 min (t₂), assuming that at the 5-min point (t₂) the measurement signal is still between the lower and upper limits (L and H) of the calibration curve 32.2 formed for that moment in time t₂. Thus, because the calibration curves 32.1, 32.2 defined for different moments in time t₁, t₂ can have dynamic, i.e. usability ranges differing from each other (at least partly) and thus also the criterion values L, H defining them, on the basis of which the progress of the measurement is controlled and/or information 28 concerning the sample's 19 measurement is formed can differ, this is not necessarily the case. Thus, by waiting until the end point set for the test (according to the prior art), a reliable measurement and analysis result may not be obtained at all for the sample 19 in question. Such a situation can, however, be avoided owing to the invention by performing the measurement as described above according to the invention when the test is still in the development stage and thus a reliable measurement and analysis result obtained from the relevant same sample 19. I.e., in other words, at at least one moment in time t₁, t₂ of the sample's 19 measurement is before the influence of the Hook effect on the sample's 19 measurement defined in connection with the test device's 10′ calibration.

As already stated above, as stage 1804 information 28 is formed on the basis of the measurement performed on the sample 19 and as stage 1805 the measuring device 11 reports the said information 28 concerning the sample 19 measured by the measuring device 11 and/or the progress of the measurement. As described above, the information 28 can be formed using calibration 25 concerning one or more detection areas 21, which is arranged for the test device 10′ and the sample 19 arranged to be analysed using it. The calibration 25 can be arranged for the measuring device 11, particularly if the information 28 too is formed and reported by it. In addition, the calibration 25 can be arranged for the measuring device 11 already beforehand, i.e. before the actual measurement of the unknown sample 19. Thus, the sample's 19 measurement is possible without requiring the operator to perform preparatory measures relating to the measurement to be performed. In addition, the information 28 can be said to be formed on the basis of the measurement result 23 and its formation, i.e. in connection with the measurement. The information 28 can be reported using the corresponding measuring device 11, by which the actual measurement of the sample 19 was performed. The information 28 can be, for example, the sample's 19 numerical analysis result 24.

As stage 1806, the test device's 10′ measurement procedure is terminated at the end point of the measurement of the test device 10′ set for the measuring device 11. It can be set for the measuring device 11, for example, along with the calibration 25 and is according to the invention described immediately above, i.e. dynamic, depending on the stage of development of the sample's 19 measurement the measurement's usability data is obtained and on its basis the sample's 19, for example, numerical analysis result 24 is formed.

As already explained above, in the invention it is possible to operate at even a single point in time t₁ of the measurement and thus with the calibration curve 32.1 relating to it. This is particularly the case when the calibration curve's 32.1 dynamic range is sufficiently large, i.e. it covers both low and also high concentrations. According to an exemplary embodiment, a typical implementation can, however, be, for example, such that, for example, 2-4 calibration curves are used, such as, for example t₁ (1.13 min) and t₂ (5.08 min) points in time, and possibly also, for example, t₃ (for example, at the 15-min point). In some special cases even more calibration curves can be used. This is particularly so if all the curves have different dynamic areas, or at least one that deviates from each other at some point, with which the deviation and test sensitivity (derivative's magnitude—large slope) is optimal in different curves and according to the sample's 19 concentration a suitable calibration curve 32.n is used at a suitable moment in time tₙ. The curves' usability can thus also be said to be concentration-dependent.

Thus, in short, the dynamic range of the test on the usability of the measurement is expanded, according to the invention, by performing the test at two or more different moments in time, and thus also the test's calibration for two or more different moments in time. The actual measurement of the test sample can then also be performed at a set moment of time defined by the calibration, and the test sample's concentration can be ascertained already before the test's development to its “end point”, if the measurement signal given by the sample 19 at the relevant measurement moment defined in the calibration is in the usability range (L-H) of the calibration curve 32.1 formed for the moment. In addition to the expanded dynamic range this also accelerates the giving of the test result and thus increases the user-friendliness of the measurement and measuring device to the patient and the measurement performer. Thus, the sample's 19 analysis result is formed and reported as information 28 at the first moment in time t₁, t₂,, of the sample's 19 measurement defined by the calibration 25, if the measurement variable 23 is in the value range [L, H] defined by the criterion values L, H of the set of criteria Kₜ₁, Kₜ₂.

In the context of the invention, the value interval [L, H] can be referred to as, for example, the quantification range. This refers to the range, in which the measurement result, such as, for example, a specific concentration, can by some set criterion be differentiated from the other measurement results, such as, for example, the concentrations (resolution). Other criteria defining the value interval can be, for example, customer specifications, the characteristic behaviour of a test-device batch 29, a product's/test's repeatability, deviation, resolution, etc. What is important, however, is that the dynamic ranges of the points in time t₁, t₂ used in the measurement are consecutive or partially overlapping. In other words, there are then no openings in the dynamic range of the calibration totality, which any calibration function 32.1, 32.2 used does not cover. In some cases, the dynamic range defined by the calibration could even be discontinuous, but this is not one of the invention preferred embodiments.

In some cases, as was already stated above, the lower limit L and the upper limit H of the calibration curve's usability area [L, H] can even be of equal magnitude. Such a case would be precisely such an application, in which the expanded dynamic range is not utilized, instead it is only reported to the operator that the sample's 19 concentration exceeds the dynamic range, i.e. that measurement with the relevant sample is not usable. For example, at the moment in time t₁ a measurement can be made to test if the measurement result is >H. If it is, the measurement is not continued, instead it is reported that the measurement result exceeds the test's dynamic range. If the measurement result at time t₁ is <L, (i.e. if L=H) a situation will not arise, in which a numerical result would never be given at time t₁—then H, i.e. the upper limit can be “forced erroneously” to be the same as L. I.e., if it was wished to give a numerical result, L would not be the same as H, but by setting them to be the same, the desired behaviour of the measuring device 11 is obtained from the same algorithm without a need for a different algorithm for this special case, then the test is allowed to develop further and is measured again at the moment in time t₂, when a function adaptation and L and H values made for this latter moment in time are applied. If the result is between L-H, the result/signal is placed in the function and the operator is given the result (in concentration units). L and H can be defined, for example, either as signal levels (for example, as bits) or as concentration units after placing in the function. In principle, they can be defined behind any kind of calculation, for example, for encryption, but what is important is that they are used to tell the measuring device 11, whether it can apply the calibration function 32.1, 32.2 to give a numerical analysis result 24 (i.e. to place the measurement signal in the calibration function).

Owing to the invention, the dynamic area of the test can differ at different points in time. Thus, by performing calibration and, as a result, also the measurement itself at several different points in time t₁, t₂, owing to the invention a wider measurement range is obtained for the test. In addition to that, and even though the test's dynamic range would be same at different points in time, using the method according to the invention it is nevertheless possible to further reduce the deviation due to the measurement and possibly detect deviations taking place in the test's development (above-mentioned Hook's phenomenon, a possibly too small amount of sample, flooding of the test base, etc.).

In addition to methods for forming calibration data for the measuring device 11 and for performing measurement using the measuring device 11, the invention also concerns a corresponding measuring device 11 and computer programs 37′. The devices, particularly the measuring device 11 and also the device that is used to calibrate the test, are configured to operate according to the methods. This is also implemented in the computer programs 37′.

FIG. 15 shows an example of a measuring device 11 for measuring a sample 19 and FIG. 22 shows the measuring device 11 in rough principle on a schematic level. As basic components, the measuring device 11 includes a reader part 16, a receiver part 35, memory 14, a processor part 13, a clock counter 36, and an output part 12.2. The parts and especially their functionalities can also overlap each other.

On such can be the clock counter 36. For example, to the processor part 13 can be such embedded in. The measuring device 11 will then be without a separate special clock counter 36.

The reader part 16, which can also be called the measuring means, is equipped with at least one reader element 16.1. The reader part 16 is arranged in the reader element 16.1 to measure from the test device's 10′ indicator part 20 one or more detection areas 21 equipped with a test chemistry 26 for the sample's 19 analysis. There are numerous different ways to implement the reader element 16.1. According to one embodiment it can include an impulse part 33.1 and a response part 33.2. The impulse part 33.1 is used to send an excitation signal to the detection area 21, such as, for example, a light beam 46, which is read in turn by the response part 33.2, i.e. the detector. The impulse and response parts can also be one and the same unit, such as, for example, in measurement performed using a magnetic sensor 16′, which forms a magnetic field MF, from the change of which the measurement result is ascertained.

The test device's 10′ receiver part 35 is arranged in connection with the reader part 16. It is arranged to receive the test device 10′ in connection with the measuring device 11 for measuring the sample 19. Thus, the excitation effects and the response's reading take place in connection with the receiver part 35, in the influence area of which is at least one detection area 21 of the test device 10′, in connection with the sample's 19 measurement. The receiver part 35 appears outwards from the measuring device 11, for example, as a reception opening for the test strip 10.

Memory 14 is arranged in the measuring device 11 for calibration 25 concerning one or more detection areas 21 of the test device 10′. The data belonging to calibration 25 is in the memory 14, as are the measuring device's 11 operating system and the applications to be executed in it, such as, for example, the measurement program 37 according to the invention. Of these, at least the calibration 25, but possibly also the applications can be updated. The device's 11 memory 14 includes, in addition, reservations, for example, for measurement results, measurement parameters, and the control of the hardware. The measuring method used by the device 11, in addition to its factory installation, can thus be updated.

Of course a similar operation, i.e. the provision of the calibration parameters 25.1 and the related sets of criteria Kₜ₁, Kₜ₂ for the measuring device 11, can also be implemented in the form of an electric signal, for example, by downloading through a (partly wireless) data network, i.e. without any physical data storage means 34, 34′ particularly to be installed in the measuring device 11. There are then data-transmission means in the measuring device 11, (or, for example, in a computer connected to it) for downloading calibration data 25, for example, from an external server or similar. An example of this is, for instance can be said the test-device manufacturer's server, in which calibrations 25 are stored and can be downloaded test-device-batch specifically.

The processor part 13 is connected to the reader part 16 to form a measurement variable 23 from the sample 19 being analysed to be arranged in connection with the receiver part 35 arranged to the test device 10′. Information 28 on the sample 19 i.e. for example the sample's 19 analysis result 24, based on the measurement variable 23 is, in addition, arranged to be formed by the processor part 13, using the calibration 25, arranged for the sample 19 being analysed, concerning one or more detection areas 21 prearranged, for example, in the measuring device's 11 memory 14.

The clock counter 36 is for controlling the operation of the processor part 13, for performing the sample's 19 measurement. It can be used to determinate, for example, the termination time of the measuring procedure at the end point of the measurement of the test device 10′ set for the measuring device 11.

The output part 12.2 is for reporting information 28 relating to the measured sample 19.

The measuring device 11 can be connected to a computer or similar data-processing device, for example, through a data-transfer interface 17 arranged in it. In addition, there are user interface means 12 in the device 11. These now include input means (keyboard) 12.1 and output means (display) 12.2. In the measuring device 11 there is also a data-transfer interface 41 for taking the calibration parameters to the measuring device 11 using, for example, a card-form data carrier 39.

In the measuring device 11, the processor means 13 are arranged to control the operation of the measuring device 11 in such a way that by the reader part 16 is arranged to measure one or more detection areas 21 at a moment of time t₁, t₂ defined by the clock counter 36 in connection with the development of the sample's 19 measurement. At least one of the moments in time is before the preset end point of the test device's 10′ measurement. In addition, the processor means 13 are arranged to control the measurement device's 11 operation in such a way that, on the basis of the measurement performed by the reader part 16 and the calibration 25 arranged in the memory 14, the usability of the sample's 19 measurement in connection with the development of the sample's 19 measurement is defined, on the basis of which the progress of the measurement is arranged to be controlled and/or information 28 concerning the sample's 19 measurement is arranged to be formed. The processor means 13 are arranged in the measuring device 11 to perform the sub-stages of the method according to the invention.

The invention also relates to a computer-readable storage means 14′. It is arranged to store the program 37, which performed by the processor part 13 of the measurement device 11, which includes a reader part 16, receiver part 35, clock counter 36, and output part 12.2, achieve the measuring device 11 to operate, such as a measuring device 11 according to the invention. The storage means 14′ is, for example, the measuring device's 11 memory 14.

Yet another object of the invention is a computer program 37′. The computer program 37′ includes instructions, which achieve the measurement device 11 implement the method according to the invention, when the computer program 37′, more particularly the measuring program 37 is executed in the programmable measuring device 11.

The calibration measurements can be performed, for example, by a corresponding measuring device 11, by which the measurement itself (by the end user) takes place. Here the difference may be only in the software. In the calibration measurement the result can be, for example, an LSB value (the result of an AD converter, i.e. a linear of, for example, amount of the magnetic particles). Of course the same measurement procedure is also possible using, for example, an optical measurement methods, i.e. the invention is not restricted to the magnetic particles referred as an example. The adaptations and the formation of the calibration functions and limits L, H can take place on a computer using a suitable program.

In addition to the use of the invention eliminating the Hook effect, owing to it the upper end of concentrations can also be measured with a short development time t₁ and with a longer development time t₂ the lower end of concentrations can be more reliably measured. The lower and upper limits Lₜ₁, Lₜ₂ and Hₜ₁, Hₜ₂ in the previous example depict this well. In other words, with a short development time, for example, the deviation caused by the test strips 10 is taken into account, particularly at low concentrations. Thus, the low signals' low LSB values, and the deviation appearing in them, limits the range of usability of the calibration curves 32.1, 32.2 in the earlier moments of time of the measurement.

The deviation is caused, for example, by the test's manufacturing. It can be seen from the calibration measurements' mean value that the result of high concentrations remains hidden under lower concentrations.

When using the test according to the example, the deviation can in reality also be at the one-minute point, due to the test's manufacturing. Instead of deviation, one can speak, for example, of the total error, i.e. by how many precent the mean value deviates from that expected. To this is added the mean deviation using a suitable reliability range, i.e. +1.65*SD, i.e., for example at a 90-% reliability range. A specific acceptance criterion exists for this, i.e. it must be, for example, less than 26%. The actual mean deviation does not necessarily drop, but the results are nevertheless more valid if the mean value is closer to that expected. In any event, purely deviation (SD not even % CV) will not necessarily tell the truth, if the mean value is totally wrong.

In the title invention, in which only the test line 21 itself is important, the method is independent of how many reactions independent of each other take place in the test device 10′. Thus, the idea according to the title invention can equally also be applied in multiplexing tests, in which there can be several detection areas in the same test device 10′. Because a control area is not necessarily needed at all in the invention, this substantially simplifies both the test-device implementation and also the measuring device 11.

At several points in the present application reference is made by way of example to a test strip 10 or a corresponding group. More generally however, instead of a test strip 10 one can speak of a test environment or a test set-up. The test environment or set-up can also be called even more generally the test device 10. The reagents are placed on the test device 10′ in such a way that, when a test reaction occurs, the measurement signal increases as a function of time. Some, but not restrictive, examples of test devices 10′ can be said a lateral-flow assay 10* and an SPR (surface plasmon resonance) device. Or more generally, all sandwich-assay test devices 10″.

Referring to FIGS. 16a-16c, for example, in the case of the later-flow assay 10* given as an embodiment example, test device's 10′, i.e. now the test-strip's 10 basic parts are a receiver part 43 (sample pad) for receiving the sample 19. From there it moves as a capillary flow 47 to the indicator part 20 through a conjugate pad 45 or similar containing test chemistry, in which the desired bonding takes place. The indicator part 20 has at least one detection area 21, i.e. now a test-line 21′. After the indicator part 20 is a collector part 44, such as, for example, a suction pad. The figure's embodiment also shows a control line 22, which the operation according to the title invention does not necessarily even need. The test's parts are arranged on a porous membrane 48.

The description of the test device's 10′ parts can be extended to the side of micro-fluidistics. Then the porous material characteristic of lateral-flow assays is replaced with microchannels. A porous material too contains these same microchannels, because it is porous—but micro-fluidistics generally refers to intentionally and manageably made channels. Of course, there is much that is manageable in the manufacturing process of a porous material too, but the channels are often random in shape and dimension, in addition to which there is a relatively wide deviation in their shapes and dimensions.

For example, the SPR (surface plasmon resonance) implementation can also work in a cuvette, i.e. the test device 10′ then also lacks a receiver part 43, a capillary flow 47, and also a collector part 44. The cuvette's bottom then corresponds to the test line, i.e. acts as the detection area.

Sandwich is a well-established name for a test type, in which two binding molecules are used, of which at least one bonds specifically to the molecule 30 being examined. The molecule being examined bonds between these two binding molecules. Often the one binder is immobilized, for example, as a test line 21′ on the cuvette's bottom or in the porous material and the other binder is free in a solution. Thus, the formation of the sandwich depends on the amount of molecules 30 being examined contained in the sample 19. Sometimes the immobilized binder can be “immobilized”, for example, in short-fibre cellulose, which “floats” as a suspension.

It is central in terms of the invention that the reaction is not momentary, but that it can be followed for at least seconds,—preferably for minutes, such as, for example, 30 seconds—15 minutes, more particularly 1-10 minutes, and even more particularly 1-5 minutes. The invention is based on the observation that at different moments in time the mean speed of reaction is different at different concentrations, and thus concentrations exceeding the measurement range can be identified already at an early stage in the reaction (and a different standard curve used on them than that used on samples with a lower concentration—or report that the result exceeds the measurement limit).

In addition, in the present application at the calibration data 25.1 reference is made by way of example to the calibration function's 32.1, 32.2 values, mainly due to understandability reasons. One skilled in the art will, however, understand that in reality the calibration data 25.1 can include, for example, by the fit used to form the calibration function, such as, for example, Hill's function coefficients (calibration coefficients) for different measurement's moments in time t₁, t₂. They are taken to the measuring device 11 along with the sets of criteria Kₜ₁, Kₜ₂ (limits L, H and possible categorization A-C) and the measurement moment data t₁, t₂. The measuring program 37 arranged in the measuring device 11 is then arranged to use this fit function applied to the calibration parameters taken, and to form on their basis the calibration curves 32.1, 32.2 used in the measurement at different moments in time t₁, t₂. The categorization data, i.e. how to operate in each situation, depending on on the relation of the measurement results 23 to the criterion values L, H set for each moment in time t₁, t₂, can also be built into the measuring program 37.

Calibration functions are referred to in connection with the invention. These can also be understood as purely response curves for different points in time. The response curve can have different functions depending on, for example, the base and the desired dynamic range. The Hill's function, for example, or also simply calibration data presented in a table form, can be used for the lateral-flow assays described as an embodiment example in the present application. A simple way to perform the method according to the invention is to compare the measurement signal to the response curves defined at the predefined time points t₁, t₂. Thus, in principle owing to the calibration method according to the invention a development curve as a function of time is defined for each concentration. The measurement can then be compared to a previously known curve with the aid of the (time dependent) response curves. The measurement, described above as an embodiment example, of two time points t₁, t₂ is a special case of this and thus the number and selection of the time points can be performed very freely, taking the invention's basic idea into account.

It should be understood that the above description and the related figures are only intended to illustrate the present invention. The invention is thus not restricted to the embodiments described above or defined in the Claims, instead many different variations and adaptations of the invention, which are possible within the scope of the inventive idea defined in the accompanying Claims, will be obvious to one skilled in the art.

1-22. (canceled)

23. A method for measuring a sample comprising:
providing a calibration to a measuring device to measure the sample to be analysed, which calibration comprises:
calibration data for forming at least one calibration function at at least one moment in time in connection with a development of a measurement of the sample in at least one detection area arranged on an indicator part of a test device, and comprising test chemistry for analysing the sample; and
a set of criteria relating to at least one calibration function, which set of criteria is formed for at least one moment in time in connection with the development of the measurement of the sample which corresponds the calibration function in question, and the set of criteria comprises at least one or more numerical criterion value defined from the calibration function corresponding at the relevant moment in time, to categorize the measurement into at least two categories, which is based on one or more numerical value ranges defined from the calibration function on the basis of the one or more numerical criterion value, in which one or more value ranges a derivative of the calibration function is continuously positive;
measuring the sample to be analysed in connection with the development of the measurement of the sample using the measuring device, from at least one detection areas arranged on the indicator part of the test device, and comprising test chemistry for analysing the sample;
defining on the basis of the measurement and the numerical value ranges an usability of the measurement of the sample in connection with the development of the measurement of the sample, on the basis of which a progress of the measurement is controlled and/or information concerning the measurement of the sample, is formed, which comprises an analysis result of the sample in value ranges defined by one or more criterion values belonging to the sets of criteria, in which the derivative of the calibration function is continuously positive; and
reporting the information concerning the measured sample.

24. The method according to claim 23, wherein the categorization defined by the value ranges, relating to the usability of the measurement of the sample and/or the control of the progress of the measurement defined at the moment in time in connection with the development of the measurement of the sample, comprises at least one of the following:
at least one value range, by which the measurement of the sample is rejected; and
at least one value range, on the basis of which the sample is measured, to define the analysis result from the sample.

25. The method according to claim 24, wherein
the calibration and the set of criteria are formed for at least two moments in time, of which at least one moment in time is in connection with the development of the measurement of the sample;
the set of criteria formed for at least one moment in time in connection with the development of the measurement of the sample, belonging to the calibration, comprises at least two criterion values defined from the calibration function corresponding the relevant moment in time, to categorize the measurement at the moment in time in at least three categories; and
the categorization is based on the value ranges defined from the calibration function on the basis of criterion values, on the basis of which the usability of the measurement of the sample is defined in connection with the development of the measurement of the sample, and/or the control of the progress of the measurement, which comprises at least one of the following:
at least one value range, by which the measurement of the sample is rejected; and
at least one value range, by which either
the analysis result of the sample is formed as the information, or
the measurement of the sample is continued at the next moment in time defined by the calibration.

26. The method according to claim 25, further comprising measuring the sample to be analysed using the measuring device from at least one detection areas, thus forming a measurement variable at a moment in time in connection with the development of the measurement of the sample and, on the basis of the set of criteria,
rejecting the measurement of the sample, if the measurement variable formed by the measuring device on the basis of the measurement of a detection area exceeds the criterion value belonging to the set of criteria, at the corresponding moment in time;
forming and reporting the analysis result of the sample as information, if the measurement variable formed by the measuring device on the basis of the measurement of the detection area is in one or more value ranges defined by the set of criteria corresponding the relevant moment in time; and/or
continuing the measurement at the next moment in time defined by the calibration, if the measurement variable formed by the measuring device on the basis of the measurement of the detection area is less than the criterion value belonging to the set of criteria.

27. The method according to claim 24, wherein the one or more criterion values, formed on the basis of a calibration function, belonging to the set of criteria, on the basis of which the progress of the measurement is controlled and/or the information concerning the measurement of the sample is formed, deviate at least partly from each other between different moments in time.

28. The method according to claim 25, further comprising forming and reporting the analysis result of the sample as the information at the first such moment in time, when the measurement variable is in the value range defined by the criterion values of the set of criteria.

29. The method according claim 23, further comprising arranging the calibration, together with the sets of criteria and the calibration data, to the measuring device in the form of the factory calibration of the manufacturing batch of the test devices.

30. The method according to claim 23, further comprising measuring at least one detection areas in connection with the development of the measurement of the sample at at least one moment in time defined in the calibration, of which at least one moment in time is before the influence of the Hook effect on the measurement of the sample, defined in connection with a calibration of the test device.

31. The method according to claims 26, further comprising measuring the measurement variable in connection with the development of the measurement of the sample at a moment in time when the measurement variable measured from the sample is increasing in value.

32. The method according to claim 23, wherein the test device comprises a sandwich-assay.

33. A measuring device for measuring a sample, comprising:
a reader part comprising at least one reader element, which comprises an impulse part and a response part and which reader part is arranged to measure from an indicator part of a test device at least one detection areas comprising test chemistry for an analysis of the sample;
a receiver part arranged in connection with the reader part, arranged to receive the test device in connection with the measuring device to measure the sample;
memory arranged for a calibration concerning at least one detection areas of the test device, which calibration comprises:
calibration data for forming at least one calibration function at at least one moment in time in connection with a development of the measurement of the sample; and
the set of criteria relating to at least one calibration function, which set of criteria is formed for at least one moment in time in connection with the development of the measurement of the sample which corresponds the calibration function in question, and the set of criteria comprises one or more numerical criterion value defined from the calibration function corresponding at the relevant moment in time, to categorize the measurement into at least two categories, which is based on one or more numerical value ranges defined from the calibration function on the basis of the one or more numerical criterion value, in which one or more value ranges a derivative of the calibration function is continuously positive;
a processor part connected to the reader part for forming a measurement variable from a sample to be analysed fitted to the test device, to be arranged in connection with the receiver part, and by which processor part is, in addition, arranged to be controlled an operation of the measuring device and formed information on the basis of the measurement variable using the calibration;
a clock counter to control the operation of the processor part to perform the measurement of the sample so that the reader part is arranged to measure at least one detection areas at a moment in time, defined by the clock counter, in connection with the development of the measurement of the sample, and on the basis of the measurement and numerical value ranges to define an usability of the measurement of the sample in connection with the development of the measurement of the sample, on the basis of which a progress of the measurement is arranged to be controlled and/or information, concerning the measurement of the sample, is arranged to be formed which comprises an analysis result of the sample in value ranges defined by one or more criterion values belonging to the sets of criteria, in which the derivative of the calibration function is continuously positive; and
an output part to report the usability of the measurement of the sample and/or information concerning the measured sample.

34. A method for performing the calibration of a test-device batch comprising:
providing a group of samples with a known concentration for a group of test devices selected from the test-device batch and comprising an indicator part for measuring at least one analyte from the samples;
measuring at least some of the samples in connection with a development of measurement of each sample, using the measuring device from at least one detection area arranged on the indicator part of the test device and comprising test chemistry for analysing a sample, to form development data for different concentrations of the sample;
forming the calibration of the test-device batch for the sample to be analysed on the basis of the measurements and the formed development data, comprising the calibration data concerning at least one detection area, formed for at least one moment in time in connection with the development of the measurement of the sample and to form a calibration function for the relevant moment in time;
selecting and recording at least one calibration function, the derivative of which is positive;
forming and recording a set of criteria for the measurement of the sample by defining as criterion values at least one of the following from each of the selected calibration functions: at least one measurement-range upper limit, or the corresponding data and/or at least one measurement-range lower limit, or the corresponding data;
forming an operating instruction for the measuring device and the measurement of the sample on the basis of the criterion values belonging to the set of criteria, comprising at least one of the following:
ending the measurement of the sample at the relevant moment in time defined by the calibration, if the measurement variable determined from the sample being measured at the relevant moment in time is above the upper limit; and/or
continuing the measurement of the sample at the next moment in time defined by the calibration, if the measurement variable determined from the sample being measured at the relevant moment in time is below the lower limit; and/or
defining an analysis result of the sample, if the measurement variable is in the range between the lower limit and the upper limit;
recording the set of criteria belonging to the calibration, together with the criterion values and their corresponding operating instructions; and
recording the calibration data concerning the test-device batch into a data structure formed for the calibration to be taken to the measuring device on the basis of which calibration information concerning the sample of unknown concentration can be formed, by using the measuring device comprising the calibration.

35. The method according to claim 34, further comprising
measuring the samples as a continuous process as time progresses,
selecting to the calibration on the basis of the measurement, at least one moment in time, at which the derivative of the calibration function is continuously positive and has sufficient resolution, taking into account the measured deviation of the test devices.

36. The method according to claim 34, further comprising forming the data structure in connection with a factory calibration of the test-device batch.
